SEQUENCE LISTING (100 sequences)

SEQ ID NO: 1 - DNA, 1176 bp, Caulobacter vibrioides
  CDS (1)..(1173): Codon-optimized oligonucleotide
atgcgcactcaggtggctatcgtaggagcaggcccagctggcctgttcttgggccatctcctccgtcaagctggtgtggacgtcgtgattctggaacgcaaagaccgtgcttatgtcgaaggccgagttcgggctggtgtcctggaacgtatcacggtggagctgatggagcgtcttggtgtggatgagcgaatgcgccgagagggcttggtgcatgctggcgctaatcttgcgtctgatggcgagatgttccgtatcgacatggcagagctcacgggtggttccaccgtcatggtttacggccaacaggaggtgatgaaggacctgtttgatgcagcagagcagcgcgatctgcgaattgtctttgacgccgatgcagtgcgtctgcacgatgtggaaggcgaacgtcctcacatcacctggcgcaaagacggggcagaacaccgcctggactgcgatttcattgccggctgcgacggctaccacggagtttctcgtgcgaccattcccgataaggttctcaagaccttcgaacgggtgtatccctttgggtggttgggaatcctggctgaagcacctccgtgtgaccacgagttgatctactcgaaccatgatcgcggttttgccctggcgtcgatgcgctcaccgacacgctcccgctattacgtgcagtgctcactcgacgatcgcctcgaggattggtccgatgaacggttctgggatgaagtttcggttcgcctgggaccggaagcagccgctcggatcgttcgcgcaccttccttcgagaagagcattgccccacttcgctccttcgtttccgagcctatgcggtatggccgccttttcctcgcgggtgatgcggctcatatcgttccacccactggagcgaaagggatgaacttggccgtatcagacgtcatcatgctgtccgaagccctggtcgaacactaccacgaacgctcttccgctggtatcgatggttacagcgcacgtgcacttgcccgcgtctggaaggcggagcgtttcagctggtggtttacctcccttactcaccgcttcccagaccaggacggcttcgaccgcaagatgcaagtcgccgaattggcatacatcaagggttctcgcgctgcccaggtcaccctggcggagaactacgtaggactgccactcgtataa

SEQ ID NO: 2 - protein, 391 aa, Caulobacter vibrioides
MRTQVAIVGAGPAGLFLGHLLRQAGVDVVILERKDRAYVEGRVRAGVLERITVELMERLGVDERMRREGLVHAGANLASDGEMFRIDMAELTGGSTVMVYGQQEVMKDLFDAAEQRDLRIVFDADAVRLHDVEGERPHITWRKDGAEHRLDCDFIAGCDGYHGVSRATIPDKVLKTFERVYPFGWLGILAEAPPCDHELIYSNHDRGFALASMRSPTRSRYYVQCSLDDRLEDWSDERFWDEVSVRLGPEAAARIVRAPSFEKSIAPLRSFVSEPMRYGRLFLAGDAAHIVPPTGAKGMNLAVSDVIMLSEALVEHYHERSSAGIDGYSARALARVWKAERFSWWFTSLTHRFPDQDGFDRKMQVAELAYIKGSRAAQVTLAENYVGLPLV

SEQ ID NO: 3 - DNA, 1173 bp, Sinorhizobium meliloti
  CDS (1)..(1170): Codon-optimized oligonucleotide
atgcgcacccaagtggtcatcatcggctcaggaccgtctggccttcttctgggacagcttctgaccgaggcagggatcgcaaacgtcatcctcgatcgcgctaccaaggcccacattctcgggcgagttcgcgctggagtgttggaacagggcaccgttcgccttatggaagaggctggttgtggtgcgcgaatgcacgcagaaggactgccacacgacggcttttcgctggcattcgacggtcgggatcaccgcattgacctgttcggcctgactggtggccgtcgcgtaatgatctatggtcagacggagctgacacgggacctgatggatcatcgcgagcgggttggtgcactctccatctacgaagcggctaacgtcatgccccgcgatttcgatggacggacacctcacgttgcgtatgagaaggacggtattgcgcaacgcattgactgcgacttcatcgccggctgtgacggcttccatggcgtgtcccgtcgttccctgccagagaaagccatccggaatttcgagaagatctacccatttggatggctcggcatcctggcagatgtcccaccggtcgatcatgaactcgtgtacgctaatcacccacgcgggtttgcattgtgctctatgcgttcccatacccgttctcgctactacatccagtgccctttggaggaaaagatcgaagattgggatgatcagcgcttctgggatgaactccgccgtcgtctgcctgcgcaccatgcggagcgcgtggtaaccggtccgagcttcgagaagtccattgcacccttgcgttcgttcgttgccgaaccaatgcgcttcaaccgcctgttcttggcgggcgatgctgcccacattgtcccacctaccggtgctaaaggccttaacctcgctgcttccgacgtccactacctgtttgaggggttgctcgaacactaccaggatcgaagcaacgcaggcatcgacgcctattccgctcgcgcactggctcgtgtgtggaaagccgttcgcttcagctggtggatgacgactatgcttcaccgttttcccgaaacctccgactttgaccagcgcattcaagaggccgaactggactatctcacccactcacgagctgccgcaactgcacttgcggaaaactacgtgggtctgccgttctaa

SEQ ID NO: 4 - protein, 390 aa, Sinorhizobium meliloti
MRTQVVIIGSGPSGLLLGQLLTEAGIANVILDRATKAHILGRVRAGVLEQGTVRLMEEAGCGARMHAEGLPHDGFSLAFDGRDHRIDLFGLTGGRRVMIYGQTELTRDLMDHRERVGALSIYEAANVMPRDFDGRTPHVAYEKDGIAQRIDCDFIAGCDGFHGVSRRSLPEKAIRNFEKIYPFGWLGILADVPPVDHELVYANHPRGFALCSMRSHTRSRYYIQCPLEEKIEDWDDQRFWDELRRRLPAHHAERVVTGPSFEKSIAPLRSFVAEPMRFNRLFLAGDAAHIVPPTGAKGLNLAASDVHYLFEGLLEHYQDRSNAGIDAYSARALARVWKAVRFSWWMTTMLHRFPETSDFDQRIQEAELDYLTHSRAAATALAENYVGLPF

SEQ ID NO: 5 - DNA, 1173 bp, Rhodopseudomonas palustris
  CDS (1)..(1170): Codon-optimized oligonucleotide
atgcgcactcaggtggcaatcattggtgccggtccatccggtctgcttctcggacagctccttcacaagtacgggatcgacgccgtcatcgttgagcgcaaagatcccgattacgtgctttcccgcatccgtgcaggcgttctggagcagggcatggttgacctcctggacgaagctggcgtaagcgctcggttgcatcaggaagcgcttgttcacggtggtttcgaaatcgcgttcgcaggccaacggcatcccattgatctgcgtggtgcaaccggaggcaagtctgtcaccgtctatggtcagaccgaggtaacccgagatctcatggaggctcgatctgcagctggccttacgacgatctatgacgctgcggatgtttcgctgcatgactttgaaggcgcacaccctaaggtgcgttacgtcaaagacggcaccactcgcgagatcgtgtgcgatttcattgcgggatgtgacgggttccacggaatttcgcgccaatctgtgccagcgtctgccgttcagtccttcgaacgcgtgtatccgttcggctggttgggacttctgtccgatacccctcccgtcagcccagaactgatctacgtcaaccacgaccgaggctttgctctctgctcgatgcgctccatgcaccgttcacggtactacgtccaatgccctttgaccgacgatgtcgctgattggtctgatgatcgcttctgggacgaactgaagtcccgcctcgacccggaaacagcggggaaactcgtgacaggcccgtcaatcgagaagtcaatcgcaccactgcgttcctttgtcgctgaaccaatgcgctttggtcgccttttcctggccggtgatgccgctcacattgttccgcctaccggagcaaagggcttgaatctggcggcttcagacgtgtactacctgagccgtgcaatgcgggagtactatgccgagaagtccgaagccggtatcgacgcttactcggcatccgccttgcgccgtgtttggaaggcggaacgtttctcctggtggatgacttctcagctgcaccgctttccagattccgacgcattcagccaacgcatccagactgctgagctcgattatctggtgaactccaaagcagccattacctccttggccgagaactacgtaggcctgccatactaa

SEQ ID NO: 6 - protein, 390 aa, Rhodopseudomonas palustris
MRTQVAIIGAGPSGLLLGQLLHKYGIDAVIVERKDPDYVLSRIRAGVLEQGMVDLLDEAGVSARLHQEALVHGGFEIAFAGQRHPIDLRGATGGKSVTVYGQTEVTRDLMEARSAAGLTTIYDAADVSLHDFEGAHPKVRYVKDGTTREIVCDFIAGCDGFHGISRQSVPASAVQSFERVYPFGWLGLLSDTPPVSPELIYVNHDRGFALCSMRSMHRSRYYVQCPLTDDVADWSDDRFWDELKSRLDPETAGKLVTGPSIEKSIAPLRSFVAEPMRFGRLFLAGDAAHIVPPTGAKGLNLAASDVYYLSRAMREYYAEKSEAGIDAYSASALRRVWKAERFSWWMTSQLHRFPDSDAFSQRIQTAELDYLVNSKAAITSLAENYVGLPY

SEQ ID NO: 7 - DNA, 1179 bp, Bradyrhizobium diazoefficiens
  CDS (1)..(1176): Codon-optimized oligonucleotide
atgcgtactcaggtgggaatcgtgggagccggaccagccggtctgctcctctcgcacatgctgtatctgtccggcattgagtcgatcatcatcgagtcacgttcgcaggcggaaatcgagcagaccatccgcgccggggtactggaacagtctacggttgacctgatgaccgagattggggctggcgatcgcatgaagcgggaaggctttgtccatggtggctttgagctccgctttgcagggcatgggcaccggatcgatctgcagaaccttgctaatggccgcacgatcacagtctatccacagcacgaagtcctcaaggacctgattgccttgcgcttgcgtactggtggtcaaatccactttgaggctaaagctacctccattgacggtctgaccagcgatcagccagttgtgcgcttcattaccaagcaaggtgaaacccgtgaattgtcctgcgactttgtggctggctgtgacggtggctatggagcctcacgagctgcgattccggaggatctggttcgccgcgattacttccgagtgtacccgttcggttggttcggcatcctggcaaaagccccaccctcatccgaagagctcatctacgcacatcacgaccgcggattcgcgctcatcagcacacgctctccgaatatccagcgcatgtacttccagtgcgcacctaccgattccgttgacaactggagcgatgatcgcatttggaacgagttgcagactcgggttggtggcgatggcttcgagctcaagactggccctatcttccagaagggtatcattcctcttcgctccttcgtttgcgaacccatgcaacacggacggcttttccttgcaggcgatgctgcacactctgtccctcccacaggtgcaaaaggcctcaacctggcagctgcagacgtgtacgtccttgcccgtgcacttgcgagctactacgctaagcgatccaccaccctgttggacgcgtattcctctaccgcattgcgtcgcgtgtggcgtgcccaacacttctcctggtggatgacctctatgctgcattggtttcacgaaggaaccgaattcgacctgaagcgtcaactggcggagctcgaattggtcacgtcctccaaagcggctgcaaccactctggccgaaaactacgtaggcatgccacttgcttaa

SEQ ID NO: 8 - protein, 392 aa, Bradyrhizobium diazoefficiens
MRTQVGIVGAGPAGLLLSHMLYLSGIESIIIESRSQAEIEQTIRAGVLEQSTVDLMTEIGAGDRMKREGFVHGGFELRFAGHGHRIDLQNLANGRTITVYPQHEVLKDLIALRLRTGGQIHFEAKATSIDGLTSDQPVVRFITKQGETRELSCDFVAGCDGGYGASRAAIPEDLVRRDYFRVYPFGWFGILAKAPPSSEELIYAHHDRGFALISTRSPNIQRMYFQCAPTDSVDNWSDDRIWNELQTRVGGDGFELKTGPIFQKGIIPLRSFVCEPMQHGRLFLAGDAAHSVPPTGAKGLNLAAADVYVLARALASYYAKRSTTLLDAYSSTALRRVWRAQHFSWWMTSMLHWFHEGTEFDLKRQLAELELVTSSKAAATTLAENYVGMPLA

SEQ ID NO: 9 - DNA, 1179 bp, Cupriavidus metallidurans
  CDS (1)..(1176): Codon-optimized oligonucleotide
atgcgcactcaggttggtatcattggagctggtccagcaggcttgctcctttcccatctcctgcacctcaagggtatcgattctgtcgtcatcgaatctcggacacgcgaagagatcgagtccacgattcgggcgggtgtactggaacagggcaccatggacctcttgcaggatgttggccttgggaatcgcatggtcgccgagggcgcacttcaccaaggcttcgaattgacctttgagggcgaacggcatcgtatcgatctcaccgacctgacgggccatgcgatcaccgtatacgcacagcacgaagtgatcaaggatcttgtggcagcacgagtggcagctggtgggaaactggttttcggcgtttcgaacacttctctccacgatgtggaatccaccaaaccgtcgattcgctacgagaaggacgacgacacgtgcgaaattcactgcgacttcgtcatcgggtgtgatggctcacaaggaccatcacgctctgcaattcccgaagcagtccgcaaagaccatcagcgcttgtacccctttggctggtttggcattctggtcgaagctccgccttcatccgacgagctgatctacgcacgacatgaccgcggatttgccctgatctcgactcgtagccctaccgtccaacgtatgtacttccagtgcgatccacgggatagcgttgagaactggtccgacgatcgcatctggtccgagctccacgcacgattggatcaagccgatggttggcgtgtaactgagggccgcattttccagaagaacatcgttggtatgcgctccttcgtgagcaatgtgatgcagcacggtcgcctgtttctggctggggattctgcccacatcgttccgcctactggcgcgaaaggaatgaacctcgccgtgaacgatgtcaagatcctggctgaaggtctggactccttctacaagaacggtaccgaggacaagctgaatgcgtataccgccaccgccctgcagcgtatctggcgtgcggagcacttctcctggtggatgacctccatgttgcaccgcttcgctgatgcgaccccattcgaccagcaacttcaggtgtccgaactgcgctatgtcacctcgtcccgtgctggcgctacagctcttgccgagaactatgtgggaatggttggactgagccactaa

SEQ ID NO: 10 - protein, 392 aa, Cupriavidus metallidurans
MRTQVGIIGAGPAGLLLSHLLHLKGIDSVVIESRTREEIESTIRAGVLEQGTMDLLQDVGLGNRMVAEGALHQGFELTFEGERHRIDLTDLTGHAITVYAQHEVIKDLVAARVAAGGKLVFGVSNTSLHDVESTKPSIRYEKDDDTCEIHCDFVIGCDGSQGPSRSAIPEAVRKDHQRLYPFGWFGILVEAPPSSDELIYARHDRGFALISTRSPTVQRMYFQCDPRDSVENWSDDRIWSELHARLDQADGWRVTEGRIFQKNIVGMRSFVSNVMQHGRLFLAGDSAHIVPPTGAKGMNLAVNDVKILAEGLDSFYKNGTEDKLNAYTATALQRIWRAEHFSWWMTSMLHRFADATPFDQQLQVSELRYVTSSRAGATALAENYVGMVGLSH

SEQ ID NO: 11 - DNA, 1176 bp, Rhodococcus sp.
  CDS (1)..(1173): Codon-optimized oligonucleotide
atgcgtacccaagtggccatcattggagcgggtccagctgggctgctgctcagccacctcctggatgaacagggaatcgactcaatcctgatcgaatctcgcactcaggaatacgttctgtcacgcatccgtgccggtgtcctggagcactccacggtgcaacttctggatgagcatgggttgggtgagcgcctgcatcgcgaaggtgatgaacatcgcggcatctacttgcagtggcccgaagaacgacaccacatcgacttccgggacctggtcgatcgttccgtctgggtgtatggtcagaccgaggtgacaaaggatctggtcgtcgcacgcgagaaagcgggtcaacagatctactacgatgtgtccgacaccgcgcttcacgacgtagaatccgacgcaccctacgttaccttcactgacgcatcgggcaatgcggttcgcattgatgcaaccgttgttgcgggctgtgatggctctttcggtccatcacgggctgcaatgcctgactcggttcgtaacacctgggagcgtgtgtacccatattcctggttgggcgtgcttgcagatgtggctccttctaccgacgagctgatctatgcctggcatcaggacggttttgcaatgcactccatgcgatcctcgaccgtttctcgcctgtacctccaggttcctaacgggactgacattgacacctggtccgacgaccgcatctgggatgctctggccctccgtcttggacacggacaagatggctggaccctgaatcccggcccgattaccgagaagtcggtgttgccaatgcgctcttacgtccagactccaatgcgccatggcaacctttatctggctggtgatgcagctcacatcgtcccgcctactggcgctaagggtctgaacctggctgtagcagatgtcgcactcctcgcaccagccttggcgcaaaagctcaaaggcaacgactcccgtgccgcggatagctacagcgatgatgccttgcgacgggtatggcgctgcacccacttcagctggtggatgacgacgatgcttcacacaggagatgacccgtttgatgcccagctccagctttcccagctcaagtgggtcgcatcctccgaagccggagctatgggcttggctgagaactacgctggccttccgattggcttctaa

SEQ ID NO: 12 - protein, 391 aa, Rhodococcus sp.
MRTQVAIIGAGPAGLLLSHLLDEQGIDSILIESRTQEYVLSRIRAGVLEHSTVQLLDEHGLGERLHREGDEHRGIYLQWPEERHHIDFRDLVDRSVWVYGQTEVTKDLVVAREKAGQQIYYDVSDTALHDVESDAPYVTFTDASGNAVRIDATVVAGCDGSFGPSRAAMPDSVRNTWERVYPYSWLGVLADVAPSTDELIYAWHQDGFAMHSMRSSTVSRLYLQVPNGTDIDTWSDDRIWDALALRLGHGQDGWTLNPGPITEKSVLPMRSYVQTPMRHGNLYLAGDAAHIVPPTGAKGLNLAVADVALLAPALAQKLKGNDSRAADSYSDDALRRVWRCTHFSWWMTTMLHTGDDPFDAQLQLSQLKWVASSEAGAMGLAENYAGLPIGF

SEQ ID NO: 13 - DNA, 42 bp, Artificial Sequence
  Synthetic oligonucleotide
tattaattaaatgcgcgttttaattattgataattatgattc

SEQ ID NO: 14 - DNA, 45 bp, Artificial Sequence
  Synthetic oligonucleotide
ttgcggccgcttgtttaaacctccttacagaaaaatggttgggcg

SEQ ID NO: 15 - DNA, 33 bp, Artificial Sequence
  Synthetic oligonucleotide
aaatttaaacctcctttacagaaaaatggttgg

SEQ ID NO: 16 - DNA, 28 bp, Artificial Sequence
  Synthetic oligonucleotide
ggaggtttaaacaagcggccgcgatatc

SEQ ID NO: 17 - DNA, 35 bp, Artificial Sequence
  Synthetic oligonucleotide
aggaggtttaaatttatgcgcactcaggtggctat

SEQ ID NO: 18 - DNA, 35 bp, Artificial Sequence
  Synthetic oligonucleotide
cttgtttaaacctccttatacgagtggcagtccta

SEQ ID NO: 19 - DNA, 31 bp, Artificial Sequence
  Synthetic oligonucleotide
gctatcaaaacattcggcacattggttttcc

SEQ ID NO: 20 - DNA, 29 bp, Artificial Sequence
  Synthetic oligonucleotide
ggaagatgcgtgatctgatccttcaactc

SEQ ID NO: 21 - DNA, 24 bp, Artificial Sequence
  Synthetic oligonucleotide
ttacagaaaaatggttgggcgcaa

SEQ ID NO: 22 - DNA, 22 bp, Artificial Sequence
  Synthetic oligonucleotide
atgcgcactcaggtggctatcg

SEQ ID NO: 23 - DNA, 342 bp, Artificial Sequence
  Synthetic oligonucleotide
tacgtacctgcaggtagcgtgtcagtaggcgcgtagggtaagtggggtagcggcttgttagatatcttgaaatcggctttcaacagcattgatttcgatgtatttagctggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaaaggaggatctaaattatgaataatataaaaggaggaattaattaa

SEQ ID NO: 24 - DNA, 36 bp, Artificial Sequence
  Synthetic oligonucleotide
accatttttctgtaatacgtacctgcaggtagcgtg

SEQ ID NO: 25 - DNA, 35 bp, Artificial Sequence
  Synthetic oligonucleotide
cacctgagtgcgcatttaattaattcctcctttta

SEQ ID NO: 26 - DNA, 29 bp, Artificial Sequence
  Synthetic oligonucleotide
gcttgttagatatcttgaaatcggctttc

SEQ ID NO: 27 - DNA, 18 bp, Artificial Sequence
  Synthetic oligonucleotide
ggaggtttaaacaagcgg

SEQ ID NO: 28 - DNA, 27 bp, Artificial Sequence
  Synthetic oligonucleotide
aatttagatcctcctttggacttcgtg

SEQ ID NO: 29 - DNA, 36 bp, Artificial Sequence
  Synthetic oligonucleotide
aggaggatctaaattatgcgtactcaggtgggaatc

SEQ ID NO: 30 - DNA, 32 bp, Artificial Sequence
  Synthetic oligonucleotide
cttgtttaaacctccttaagcaagtggcatgc

SEQ ID NO: 31 - DNA, 36 bp, Artificial Sequence
  Synthetic oligonucleotide
aggaggatctaaattatgcgcactcaggtggcaatc

SEQ ID NO: 32 - DNA, 34 bp, Artificial Sequence
  Synthetic oligonucleotide
cttgtttaaacctccttagtatggcaggcctacg

SEQ ID NO: 33 - DNA, 36 bp, Artificial Sequence
  Synthetic oligonucleotide
aggaggatctaaattatgcgcacccaagtggtcatc

SEQ ID NO: 34 - DNA, 37 bp, Artificial Sequence
  Synthetic oligonucleotide
cttgtttaaacctccttagaacggcagacccacgtag

SEQ ID NO: 35 - DNA, 36 bp, Artificial Sequence
  Synthetic oligonucleotide
aggaggatctaaattatgcgcactcaggttggtatc

SEQ ID NO: 36 - DNA, 38 bp, Artificial Sequence
  Synthetic oligonucleotide
cttgtttaaacctccttagtggctcagtccaaccattc

SEQ ID NO: 37 - DNA, 40 bp, Artificial Sequence
  Synthetic oligonucleotide
aggaggatctaaattatgcgtacccaagtggccatcattg

SEQ ID NO: 38 - DNA, 36 bp, Artificial Sequence
  Synthetic oligonucleotide
cttgtttaaacctccttagaagccaatcggaaggcc

SEQ ID NO: 39 - DNA, 27 bp, Artificial Sequence
  Synthetic oligonucleotide
gccgggctcctggaacagtctacggtt

SEQ ID NO: 40 - DNA, 27 bp, Artificial Sequence
  Synthetic oligonucleotide
ttccaggagcccggcgcggatggtctg

SEQ ID NO: 41 - DNA, 27 bp, Artificial Sequence
  Synthetic oligonucleotide
gctggtctcctggaacgtatcacggtg

SEQ ID NO: 42 - DNA, 27 bp, Artificial Sequence
  Synthetic oligonucleotide
ttccaggagaccagcccgaactcggcc

SEQ ID NO: 43 - DNA, 27 bp, Artificial Sequence
  Synthetic oligonucleotide
gcaggcctcctggagcagggcatggtt

SEQ ID NO: 44 - DNA, 27 bp, Artificial Sequence
  Synthetic oligonucleotide
ctccaggaggcctgcacggatgcggga

SEQ ID NO: 45 - DNA, 27 bp, Artificial Sequence
  Synthetic oligonucleotide
gctggactcttggaacagggcaccgtt

SEQ ID NO: 46 - DNA, 27 bp, Artificial Sequence
  Synthetic oligonucleotide
ttccaagagtccagcgcgaactcgccc

SEQ ID NO: 47 - DNA, 27 bp, Artificial Sequence
  Synthetic oligonucleotide
gcgggtctcctggaacagggcaccatg

SEQ ID NO: 48 - DNA, 27 bp, Artificial Sequence
  Synthetic oligonucleotide
ttccaggagacccgcccgaatcgtgga

SEQ ID NO: 49 - DNA, 27 bp, Artificial Sequence
  Synthetic oligonucleotide
gccggtctcctggagcactccacggtg

SEQ ID NO: 50 - DNA, 27 bp, Artificial Sequence
  Synthetic oligonucleotide
ctccaggagaccggcacggatgcgtga

SEQ ID NO: 51 - DNA, 27 bp, Artificial Sequence
  Synthetic oligonucleotide
ctcatcttcgcacatcacgaccgcgga

SEQ ID NO: 52 - DNA, 27 bp, Artificial Sequence
  Synthetic oligonucleotide
atgtgcgaagatgagctcttcggatga

SEQ ID NO: 53 - DNA, 27 bp, Artificial Sequence
  Synthetic oligonucleotide
cgcatgttcttccagtgcgcacctacc

SEQ ID NO: 54 - DNA, 27 bp, Artificial Sequence
  Synthetic oligonucleotide
ctggaagaacatgcgctggatattcgg

SEQ ID NO: 55 - DNA, 27 bp, Artificial Sequence
  Synthetic oligonucleotide
ttgatcttctcgaaccatgatcgcggt

SEQ ID NO: 56 - DNA, 27 bp, Artificial Sequence
  Synthetic oligonucleotide
gttcgagaagatcaactcgtggtcaca

SEQ ID NO: 57 - DNA, 27 bp, Artificial Sequence
  Synthetic oligonucleotide
cgctatttcgtgcagtgctcactcgac

SEQ ID NO: 58 - DNA, 27 bp, Artificial Sequence
  Synthetic oligonucleotide
ctgcacgaaatagcgggagcgtgtcgg

SEQ ID NO: 59 - DNA, 27 bp, Artificial Sequence
  Synthetic oligonucleotide
ctgatcttcgtcaaccacgaccgaggc

SEQ ID NO: 60 - DNA, 27 bp, Artificial Sequence
  Synthetic oligonucleotide
gttgacgaagatcagttctgggctgac

SEQ ID NO: 61 - DNA, 27 bp, Artificial Sequence
  Synthetic oligonucleotide
cggtacttcgtccaatgccctttgacc

SEQ ID NO: 62 - DNA, 27 bp, Artificial Sequence
  Synthetic oligonucleotide
ttggacgaagtaccgtgaacggtgcat

SEQ ID NO: 63 - DNA, 27 bp, Artificial Sequence
  Synthetic oligonucleotide
ctcgtgttcgctaatcacccacgcggg

SEQ ID NO: 64 - DNA, 27 bp, Artificial Sequence
  Synthetic oligonucleotide
attagcgaacacgagttcatgatcgac

SEQ ID NO: 65 - DNA, 27 bp, Artificial Sequence
  Synthetic oligonucleotide
cgctacttcatccagtgccctttggag

SEQ ID NO: 66 - DNA, 27 bp, Artificial Sequence
  Synthetic oligonucleotide
ctggatgaagtagcgagaacgggtatg

SEQ ID NO: 67 - DNA, 27 bp, Artificial Sequence
  Synthetic oligonucleotide
gctggtattctggaacgtatcacggtg

SEQ ID NO: 68 - DNA, 27 bp, Artificial Sequence
  Synthetic oligonucleotide
ttccagaataccagcccgaactcggcc

SEQ ID NO: 69 - DNA, 27 bp, Artificial Sequence
  Synthetic oligonucleotide
gctggttctctggaacgtatcacggtg

SEQ ID NO: 70 - DNA, 27 bp, Artificial Sequence
  Synthetic oligonucleotide
ttccagagaaccagcccgaactcggcc

SEQ ID NO: 71 - DNA, 27 bp, Artificial Sequence
  Synthetic oligonucleotide
gctggtacactggaacgtatcacggtg

SEQ ID NO: 72 - DNA, 27 bp, Artificial Sequence
  Synthetic oligonucleotide
ttccagtgtaccagcccgaactcggcc

SEQ ID NO: 73 - DNA, 27 bp, Artificial Sequence
  Synthetic oligonucleotide
gctggttgtctggaacgtatcacggtg

SEQ ID NO: 74 - DNA, 27 bp, Artificial Sequence
  Synthetic oligonucleotide
ttccagacaaccagcccgaactcggcc

SEQ ID NO: 75 - DNA, 27 bp, Artificial Sequence
  Synthetic oligonucleotide
gctggtatgctggaacgtatcacggtg

SEQ ID NO: 76 - DNA, 27 bp, Artificial Sequence
  Synthetic oligonucleotide
ttccagcataccagcccgaactcggcc

SEQ ID NO: 77 - DNA, 27 bp, Artificial Sequence
  Synthetic oligonucleotide
gctggtcaactggaacgtatcacggtg

SEQ ID NO: 78 - DNA, 27 bp, Artificial Sequence
  Synthetic oligonucleotide
ttccagttgaccagcccgaactcggcc

SEQ ID NO: 79 - DNA, 27 bp, Artificial Sequence
  Synthetic oligonucleotide
ttggtggcagctggcgctaatcttgcg

SEQ ID NO: 80 - DNA, 27 bp, Artificial Sequence
  Synthetic oligonucleotide
gccagctgccaccaagccctctcggcg

SEQ ID NO: 81 - DNA, 27 bp, Artificial Sequence
  Synthetic oligonucleotide
ttggtgatggctggcgctaatcttgcg

SEQ ID NO: 82 - DNA, 27 bp, Artificial Sequence
  Synthetic oligonucleotide
gccagccatcaccaagccctctcggcg

SEQ ID NO: 83 - DNA, 27 bp, Artificial Sequence
  Synthetic oligonucleotide
tttgccatggcgtcgatgcgctcaccg

SEQ ID NO: 84 - DNA, 27 bp, Artificial Sequence
  Synthetic oligonucleotide
cgacgccatggcaaaaccgcgatcatg

SEQ ID NO: 85 - DNA, 27 bp, Artificial Sequence
  Synthetic oligonucleotide
ccacccgcaggagcgaaagggatgaac

SEQ ID NO: 86 - DNA, 27 bp, Artificial Sequence
  Synthetic oligonucleotide
cgctcctgcgggtggaacgatatgagc

SEQ ID NO: 87 - DNA, 27 bp, Artificial Sequence
  Synthetic oligonucleotide
ccacccggtggagcgaaagggatgaac

SEQ ID NO: 88 - DNA, 27 bp, Artificial Sequence
  Synthetic oligonucleotide
cgctccaccgggtggaacgatatgagc

SEQ ID NO: 89 - DNA, 27 bp, Artificial Sequence
  Synthetic oligonucleotide
ccaccctgtggagcgaaagggatgaac

SEQ ID NO: 90 - DNA, 27 bp, Artificial Sequence
  Synthetic oligonucleotide
cgctccacagggtggaacgatatgagc

SEQ ID NO: 91 - DNA, 27 bp, Artificial Sequence
  Synthetic oligonucleotide
ccaccctctggagcgaaagggatgaac

SEQ ID NO: 92 - DNA, 27 bp, Artificial Sequence
  Synthetic oligonucleotide
cgctccagagggtggaacgatatgagc

SEQ ID NO: 93 - DNA, 27 bp, Artificial Sequence
  Synthetic oligonucleotide
gagaacgttgtaggactgccactcgta

SEQ ID NO: 94 - DNA, 27 bp, Artificial Sequence
  Synthetic oligonucleotide
tcctacaacgttctccgccagggtgac

SEQ ID NO: 95 - DNA, 27 bp, Artificial Sequence
  Synthetic oligonucleotide
gagaacctcgtaggactgccactcgta

SEQ ID NO: 96 - DNA, 27 bp, Artificial Sequence
  Synthetic oligonucleotide
tcctacgaggttctccgccagggtgac

SEQ ID NO: 97 - DNA, 27 bp, Artificial Sequence
  Synthetic oligonucleotide
gagaacattgtaggactgccactcgta

SEQ ID NO: 98 - DNA, 27 bp, Artificial Sequence
  Synthetic oligonucleotide
tcctacaatgttctccgccagggtgac

SEQ ID NO: 99 - DNA, 27 bp, Artificial Sequence
  Synthetic oligonucleotide
gagaacatggtaggactgccactcgta

SEQ ID NO: 100 - DNA, 27 bp, Artificial Sequence
  Synthetic oligonucleotide
tcctaccatgttctccgccagggtgac

(19) United States
(12) Patent Application Publication
(10) Pub. No.: US 2022/0411831 A1
(43) Pub. Date: Dec. 29, 2022

(54) Polypeptide Having 4-Aminobenzoic Acid Hydroxylation Activity and Use Thereof

(71) Applicant: Kao Corporation, Chuo-ku, Tokyo (JP)

(72) Inventors: Kyoshiro Nonaka, Wakayama-shi, Wakayama (JP); Furnikazu TAKAHASHI, Wakayama-shi, Wakayama (JP)

(73) Assignee: Kao Corporation, Chuo-ku, Tokyo (JP)

(21) Appl. No.: 17/775,331

(22) PCT Filed: Nov. 6, 2020
(86) PCT No.: PCT/JP2020/041581

§ 371 Date: May 9, 2022

(30) Foreign Application Priority Data
Nov. 8, 2019 (JP) 2019-203523
Dec. 24, 2019 (JP) 2019-233484
Dec. 24, 2019 (JP) 2019-233485

(51) Int. Cl. C12P 13/02; C07K 14/195; C07K 1/107; C07C 229/60; C12N 15/77; C12N 1/20; C12N 9/10

(52) U.S. Cl. CPC C12P 13/02; C07K 14/195; C07K 1/1075; C07C 229/60; C12N 15/77; C12N 1/205; C12N 9/1096; C12Y 206/01; C12R 2001/15

(57) ABSTRACT

Provided are a polypeptide having excellent 4-aminobenzoic acid hydroxylation activity and a method for using the same. The present invention provides a polypeptide having 4-aminobenzoic acid hydroxylation activity, consisting of the amino acid sequence represented by SEQ ID NO: 2 or an amino acid sequence having at least 47% identity thereto, and having an amino acid residue at position 47 of the amino acid sequence represented by SEQ ID NO: 2 or a position corresponding thereto being leucine.

### FIELD OF THE INVENTION

The present invention relates to a polypeptide having 4-aminobenzoic acid hydroxylation activity and use thereof.

### BACKGROUND OF THE INVENTION

Polybenzoxazole (PBO) is known as an engineering plastic excellent in heat resistance and mechanical strength, and used in fiber materials and insulating films of semiconductor devices, etc. (Non Patent Literature 1).

A benzoxazole backbone was formed by the condensation of an o-aminophenol backbone with carboxylic acid. Hence, it is expected that 4-amino-3-hydroxybenzoic acids (4,3-AHBAs) having these functional groups in their molecules are useful as PBO monomers. In actuality, the synthesis and physical property evaluation of polybenzoxazole using 4,3-AHBA have been studied (Non Patent Literature 2).

Methods for producing compounds by microbial fermentation using renewable resources as starting materials have received attention in recent years for reduction in global environmental load, etc. For example, the microbial production and polymerization of 3-amino-4-hydroxybenzoic acid (3,4-AHBA), which is structurally similar to 4,3-AHBA, have been studied (Patent Literature 1).

As for the production of 4,3-AHBA, for example, a synthesis method of chemically reducing a nitro aromatic compound has been known so far (Patent Literature 2). A possible strategy which enables fermentative production of 4,3-AHBA by a microbial method is the hydroxylation at position 3 of 4-aminobenzoic acid (4-ABA) which can be biosynthesized within a microorganism. However, for such reaction, it has merely been reported that some 4-hydroxybenzoic acid hydroxylases have slight activity (Non Patent Literatures 3 and 4).
[Patent Literature 1] JP-B-5445453
[Patent Literature 2] JP-B-3821350
[Non Patent Literature 1] Hiroki Murase, SENI GAKKAISHI (Seni To Kogyo, Journal of Fiber Science and Technology), Vol. 66, No. 6 (2010)
[Non Patent Literature 2] Lon J. Mathias et al., Macromolecules, Vol. 18, No. 4, pp. 616-622 (1985)
[Non Patent Literature 3] Barrie Entsch et al., The Journal of Biological Chemistry, Vol. 262, No. 13, pp. 6060-6068 (1987)
[Non Patent Literature 4] Domenico L. Gatti et al., Biochemistry, Vol. 35, No. 2, pp. 567-578 (1996)

### SUMMARY OF THE INVENTION

The present invention relates to the following 1) to 7).

1) A polypeptide having 4-aminobenzoic acid hydroxylation activity, the polypeptide being selected from the group consisting of the following A) to C):

A) a polypeptide having 4-aminobenzoic acid hydroxylation activity, consisting of the amino acid sequence represented by SEQ ID NO: 2 or an amino acid sequence having at least 47% identity thereto, and having an amino acid residue at position 47 of the amino acid sequence represented by SEQ ID NO: 2 or a position corresponding thereto being leucine;

B) a polypeptide having 4-aminobenzoic acid hydroxylation activity, consisting of the amino acid sequence represented by SEQ ID NO: 2 or an amino acid sequence having at least 51′% identity thereto, and having an amino acid residue at position 201 or 222 of the amino acid sequence represented by SEQ ID NO: 2, or a position corresponding to the position 201 or 222 being phenylalanine; and

C) a polypeptide having 4-aminobenzoic acid hydroxylation activity, consisting of the amino acid sequence represented by SEQ ID NO: 2 or an amino acid sequence having at least 905 identity thereto, and having an amino acid residue at position 47, 72, 210, 294 or 385 of the amino acid sequence represented by SEQ ID NO: 2, or a position corresponding to the position 47, 72, 210, 294 or 385 being the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

2) A method for producing a mutant polypeptide having 4-aminobenzoic acid hydroxylation activity, comprising substituting an amino acid residue, the substituting is being selected from the group consisting of the following A′) to C′):

A′) in a polypeptide consisting of the amino acid sequence represented by SEQ ID NO: 2 or an amino acid sequence having at least 47% identity thereto, and having 4-aminobenzoic acid hydroxylation activity, substitution of an amino acid residue at position 47 of the amino acid sequence represented by SEQ ID NO: 2 or a position corresponding thereto with leucine;

B′) in a polypeptide consisting of the amino acid sequence represented by SEQ ID NO: 2 or an amino acid sequence having at least 51% identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and

C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

3) A method for improving 4-aminobenzoic acid hydroxylation activity, comprising substituting an amino acid residue, the substituting being selected from the group consisting of the following A′) to C′):

A′) in a polypeptide consisting of the amino acid sequence represented by SEQ ID NO: 2 or an amino acid sequence having at least 47% identity thereto, and having 4-aminobenzoic acid hydroxylation activity, substitution of an amino acid residue at position 47 of the amino acid sequence represented by SEQ ID NO: 2 or a position corresponding thereto with leucine;

B′) in a polypeptide consisting of the amino acid sequence represented by SEQ ID NO: 2 or an amino acid sequence having at least 51% identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and

C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

4) A polynucleotide encoding the polypeptide according to 1) or 2).

5) A vector or a DNA fragment comprising the polynucleotide according to 4).

6) A transformed cell comprising the vector or the DNA fragment according to 5).

7) A method for producing 4-amino-3-hydroxybenzoic acids, comprising a step of culturing the transformed cell according to 6).

### DESCRIPTION OF EMBODIMENTS

The present invention relates to a provision of a polypeptide having excellent 4-aminobenzoic acid hydroxylation activity and a method for using the same.

The present inventors found that a 4-hydroxybenzoic acid hydroxylase mutant having a particular amino acid sequence has excellent 4-aminobenzoic acid hydroxylation activity and is useful in the production of 4-amino-3-hydroxybenzoic acids.

Since the polypeptide having 4-aminobenzoic acid hydroxylation activity according to the present invention has excellent 4-aminobenzoic acid hydroxylation activity, use thereof enables 4-amino-3-hydroxybenzoic acids to be efficiently produced from 4-aminobenzoic acids.

In the present specification, the identity of an amino acid sequence or a nucleotide sequence is calculated by the Lipman-Pearson method (Science, 1985, 227: 1435-1441). Specifically, the identity is calculated by analysis with a unit size to compare (ktup) set to 2 using the homology analysis (search homology) program of genetic information processing software GENETYX Ver. 12.

In the present specification, the “position corresponding” on an amino acid sequence or a nucleotide sequence can be determined by aligning a sequence of interest and a reference sequence (e.g., the amino acid sequence represented by SEQ ID NO: 2) so as to provide the maximum homology. The alignment of amino acid sequences or nucleotide sequences can be carried out using an algorithm known in the art, and the procedures thereof are known to those skilled in the art. The alignment can be performed, for example, by using default settings of Clustal W multiple alignment program (Thompson, J. D. et al., 1994, Nucleic Acids Res. 22: 4673-4680). Alternatively, Clustal W2 or Clustal omega, a modified version of Clustal W, may be used. Clustal W, Clustal W2 and Clustal omega are available on, for example, the website of European Bioinformatics Institute (EBI [www.ebi.ac.uk/index.html]) or the DNA Databank of Japan (DDBJ [www.ddbj.nig.ac.jp/searches-j.html]) run by National Institute of Genetics. A position of the sequence of interest aligned to an arbitrary position of the reference sequence by the alignment mentioned above is regarded as a “position corresponding” to the arbitrary position.

Those skilled in the art can further finely adjust the alignment of amino acid sequences obtained as described above for optimization. Such optimum alignment is preferably determined in consideration of the similarity between the amino acid sequences, the frequency of a gap to be inserted, etc. In this context, the similarity between the amino acid sequences refers the ratio (%) of the number of positions at which identical or similar amino acid residues are present in both the sequences to the number of full-length amino acid residues when these two amino acid sequences are aligned. The similar amino acid residues mean amino acid residues which are similar in property to each other in terms of polarity or electric charge and cause so-called conservative substitution, among 20 amino acids constituting a protein. Groups consisting of such similar amino acid residues are well known to those skilled in the art. Examples thereof include, but are not limited to: arginine and lysine or glutamine; glutamic acid and aspartic acid or glutamine; serine and threonine or alanine; glutamine and asparagine or arginine; and leucine and isoleucine.

In the present specification, the “amino acid residue” means any of 20 amino acid residues constituting a protein, i.e., alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

In the present specification, the “operable linkage” of a gene to a control region such as a promoter means that the gene is linked to the control region such that the gene is expressible under the control of the control region. The procedures of the “operable linkage” of a gene to a control region are well known to those skilled in the art.

In the present specification, the terms “upstream” and “downstream” in relation to a gene refer to upstream and downstream in the direction of transcription of the gene. For example, the phrase “gene located downstream of a promoter” means that the gene resides on the 3′ side of the promoter in a DNA sense strand, and the phrase “upstream of a gene” means a region on the 5′ side of the gene in a DNA sense strand.

In the present specification, the term “original” which is used for a function, property, or trait of a cell is used for indicating that the function, the property, or the trait is indigenous to the cell. By contrast, the term “foreign” is used for indicating that the function, the property, or the trait is introduced ab extra, not indigenous to the cell. For example, a “foreign” gene or polynucleotide is a gene or a polynucleotide introduced ab extra into a cell. The foreign gene or polynucleotide may be derived from an organism of the same species as that of the cell harboring it or may be derived from an organism of different species therefrom (i.e., a heterologous gene or polynucleotide).

<Polypeptide Having 4-Aminobenzoic Acid Hydroxylation Activity>

The polypeptide having 4-aminobenzoic acid hydroxylation activity according to the present invention (referred to as the “polypeptide of the present invention”) is selected from the group consisting of the following A) to C):

A) a polypeptide consisting of the amino acid sequence represented by SEQ ID NO: 2 or an amino acid sequence having at least 47% identity thereto, and having an amino acid residue at position 47 of the amino acid sequence represented by SEQ ID NO: 2 or a position corresponding thereto being leucine;

B) a polypeptide consisting of the amino acid sequence represented by SEQ ID NO: 2 or an amino acid sequence having at least 51, identity thereto, and having an amino acid residue at position 201 or 222 of the amino acid sequence represented by SEQ ID NO: 2, or a position corresponding to the position 201 or 222 being phenylalanine; and

C) a polypeptide consisting of the amino acid sequence represented by SEQ ID NO: 2 or an amino acid sequence having at least 90% identity thereto, and having an amino acid residue at position 47, 72, 210, 294 or 385 of the amino acid sequence represented by SEQ ID NO: 2, or a position corresponding to the position 47, 72, 210, 294 or 385 being the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

The polypeptide A) is a mutant polypeptide having 4-aminobenzoic acid hydroxylation activity, which consists of a polypeptide serving as a reference, i.e., a polypeptide consisting of the amino acid sequence represented by SEQ ID NO: 2 or an amino acid sequence having at least 47% identity thereto, and in which an amino acid residue at position 47 of the amino acid sequence represented by SEQ ID NO: 2 or a position corresponding thereto is substituted with leucine.

The polypeptide B) is a mutant polypeptide having 4-aminobenzoic acid hydroxylation activity, which consists of a polypeptide serving as a reference, i.e., a polypeptide consisting of the amino acid sequence represented by SEQ ID NO: 2 or an amino acid sequence having at least 51% identity thereto, and in which an amino acid residue at position 201 or 222 of the amino acid sequence represented by SEQ ID NO: 2, or a position corresponding to the position 201 or 222 is substituted with phenylalanine.

The polypeptide C) is a mutant polypeptide having 4-aminobenzoic acid hydroxylation activity, which consists of a polypeptide serving as a reference, i.e., a polypeptide consisting of the amino acid sequence represented by SEQ ID NO: 2 or an amino acid sequence having at least 90% identity thereto, and in which an amino acid residue at position 47, 72, 210, 294 or 385 of the amino acid sequence represented by SEQ ID NO: 2, or a position corresponding to the position 47, 72, 210, 294 or 385 is substituted with any of the amino acids (a) to (e) set forth above.

In the present invention, the “4-aminobenzoic acid hydroxylation activity” means activity of catalyzing the hydroxylation of 4-aminobenzoic acids, preferably activity of catalyzing the hydroxylation at position 3 of 4-aminobenzoic acids.

The 4-aminobenzoic acid hydroxylation activity can be determined, as shown in Examples mentioned later, by culturing a microorganism producing the polypeptide of the present invention, and measuring the amount of 4-amino-3-hydroxybenzoic acid produced by HPLC or the like.

The polypeptide A) of the present invention can be produced by substituting an amino acid residue at position 47 of the amino acid sequence represented by SEQ ID NO: 2 or a position corresponding thereto with leucine, in a polypeptide consisting of the amino acid sequence represented by SEQ ID NO: 2 or an amino acid sequence having at least 47, identity thereto, and having 4-aminobenzoic acid hydroxylation activity.

In this context, the polypeptide consisting of the amino acid sequence represented by SEQ ID NO: 2 or an amino acid sequence having at least 47% identity thereto, and having 4-aminobenzoic acid hydroxylation activity is a “parent” polypeptide of the polypeptide A) of the present invention.

The parent polypeptide refers to a reference polypeptide which becomes the polypeptide A) of the present invention by introducing a predetermined mutation to the amino acid residue.

The polypeptide B) of the present invention can be produced by substituting an amino acid residue at position 201 or 222 of the amino acid sequence represented by SEQ ID NO: 2 or a position corresponding to the position 201 or 222 with phenylalanine, in a polypeptide consisting of the amino acid sequence represented by SEQ ID NO: 2 or an amino acid sequence having at least 51% identity thereto, and having 4-aminobenzoic acid hydroxylation activity.

In this context, the polypeptide consisting of the amino acid sequence represented by SEQ ID NO: 2 or an amino acid sequence having at least 51% identity thereto, and having 4-aminobenzoic acid hydroxylation activity is a “parent” polypeptide of the polypeptide B) of the present invention.

The parent polypeptide refers to a reference polypeptide which becomes the polypeptide B) of the present invention by introducing a predetermined mutation to the amino acid residue.

The polypeptide C) of the present invention can be produced by substituting an amino acid residue at position 47, 72, 210, 294 or 385 of the amino acid sequence represented by SEQ ID NO: 2 or a position corresponding to the position 47, 72, 210, 294 or 385 with the following amino acid, in a polypeptide consisting of the amino acid sequence represented by SEQ ID NO: 2 or an amino acid sequence having at least 90% identity thereto, and having 4-aminobenzoic acid hydroxylation activity:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

In this context, the polypeptide consisting of the amino acid sequence represented by SEQ ID NO: 2 or an amino acid sequence having at least 90% identity thereto, and having 4-aminobenzoic acid hydroxylation activity is a “parent” polypeptide of the polypeptide C) of the present invention.

The parent polypeptide refers to a reference polypeptide which becomes the polypeptide C) of the present invention by introducing a predetermined mutation to the amino acid residue.

In the present invention, HFM122, a polypeptide consisting of the amino acid sequence represented by SEQ ID NO: 2 (NCBI Reference Sequence: WP_010920262.1), is known as 4-hydroxybenzoic acid-3-monooxygenase (EC1.14.13.2). The 4-hydroxybenzoic acid-3-monooxygenase is an enzyme having catalytic activity of accelerating any one or both of reaction to produce protocatechuic acid by hydroxylating position 3 of 4-hydroxybenzoic acid and inverse reaction thereof, and is an enzyme which catalyzes the hydroxylation of 4-hydroxybenzoic acids (4-hydroxybenzoic acid hydroxylase).

This HFM122 was found to have 4-aminobenzoic acid hydroxylation activity by the present applicant (Japanese Patent Application No. 2018-171849).

<Parent Polypeptide of Polypeptide A)>

In the polypeptide A), examples of the polypeptide consisting of an amino acid sequence having at least 47% identity to the amino acid sequence represented by SEQ ID NO: 2, and having 4-aminobenzoic acid hydroxylation activity include a polypeptide which consists of an amino acid sequence having at least 47% identity, specifically, 47% or higher, more preferably 50% or higher, more preferably 60% or higher, more preferably 70% or higher, more preferably 80% or higher, more preferably 903 or higher, more preferably 95 or higher, more preferably 96% or higher, further preferably 97% or higher, further preferably 98% or higher, further preferably 99, or higher identity to the amino acid sequence represented by SEQ ID NO: 2, and has 4-aminobenzoic acid hydroxylation activity. Specific examples thereof include HFM388 (SEQ ID NO: 4; amino acid sequence identity to SEQ ID NO: 2: 62%, NCBI Reference Sequence: WP 010976283.1), HFM339 (SEQ ID NO: 6; amino acid sequence identity to SEQ ID NO: 2: 61%, NCBI Reference Sequence: WP_011157287.1), HFM77 (SEQ ID NO: 8; amino acid sequence identity to SEQ ID NO: 2: 51%, NCBI Reference Sequence: WP 011089160.1), HFM737 (SEQ ID NO: 10; amino acid sequence identity to SEQ ID NO: 2: 51%, NCBI Reference Sequence: WP_011519894.1), and HFMss0-1 (SEQ ID NO: 12; amino acid sequence identity to SEQ ID NO: 2: 47%, NCBI Reference Sequence: WP_027494688.1). Among them, HFM737 or HFMss0-1 is preferred from the viewpoint of the 4-aminobenzoic acid hydroxylation activity possessed by the polypeptide of the present invention.

Preferred examples of the “parent” polypeptide include a polypeptide which consists of the amino acid sequence represented by SEQ ID NO: 2, as well as a polypeptide which consists of an amino acid sequence having 90% or higher, more preferably 95% or higher, more preferably 96% or higher, more preferably 98% or higher identity to the amino acid sequence represented by SEQ ID NO: 2 and has 4-aminobenzoic acid hydroxylation activity. Other examples thereof include a polypeptide which consists of the amino acid sequence represented by SEQ ID NO: 4, SEQ ID NO: 6, SEQ ID NO: 8, SEQ ID NO: 10 or SEQ ID NO: 12, or a polypeptide which consists of an amino acid sequence having 90% or higher, preferably 95% or higher, more preferably 96% or higher, more preferably 98% or higher identity to each of these sequences, and has 4-aminobenzoic acid hydroxylation activity.

The parent polypeptide preferably has a valine residue at position 47 of the amino acid sequence represented by SEQ ID NO: 2 or a position corresponding thereto. For the polypeptide A) of the present invention, it is more preferred to substitute valine at the position 47 or the position corresponding thereto with leucine.

<Parent Polypeptide of Polypeptide B)>

In the polypeptide B), examples of the polypeptide consisting of an amino acid sequence having at least 51% identity to the amino acid sequence represented by SEQ ID NO: 2, and having 4-aminobenzoic acid hydroxylation activity include a polypeptide which consists of an amino acid sequence having at least 51% identity, specifically, 51% or higher, preferably 60% or higher, more preferably 70% or higher, more preferably 80% or higher, more preferably 90% or higher, more preferably 95% or higher, more preferably 96% or higher, further preferably 97% or higher, further preferably 98% or higher, further preferably 99% or higher identity to the amino acid sequence represented by SEQ ID NO: 2, and has 4-aminobenzoic acid hydroxylation activity. Specific examples thereof include HFM388 (SEQ ID NO: 4; amino acid sequence identity to SEQ ID NO: 2: 62%, NCBI Reference Sequence: WP 010976283.1), HFM339 (SEQ ID NO: 6; amino acid sequence identity to SEQ ID NO: 2: 61%, NCBI Reference Sequence: WP_011157287.1), and HFM77 (SEQ ID NO: 8; amino acid sequence identity to SEQ ID NO: 2: 51%, NCBI Reference Sequence: WP_011089160.1). Among them, HFM388 or HFM339 is preferred from the viewpoint of the 4-aminobenzoic acid hydroxylation activity possessed by the polypeptide of the present invention.

Preferred examples of the “parent” polypeptide include a polypeptide which consists of the amino acid sequence represented by SEQ ID NO: 2 as well as a polypeptide which consists of an amino acid sequence having 90% or higher, more preferably 95% or higher, more preferably 962 or higher, more preferably 98- or higher identity to the amino acid sequence represented by SEQ ID NO: 2, and has 4-aminobenzoic acid hydroxylation activity. Other examples thereof include a polypeptide which consists of the amino acid sequence represented by SEQ ID NO: 4, SEQ ID NO: 6 or SEQ ID NO: 8, or a polypeptide which consists of an amino acid sequence having 90, or higher, preferably 95 or higher, more preferably 96% or higher, more preferably 98 or higher identity to each of these sequences, and has 4-aminobenzoic acid hydroxylation activity.

The parent polypeptide preferably has a tyrosine residue at position 201 or 222 of the amino acid sequence represented by SEQ ID NO: 2 or a position corresponding to the position 201 or 222. The polypeptide B) of the present invention is more preferably a mutant polypeptide in which tyrosine at the position 201 or 222 or the position corresponding to the position 201 or 222 is substituted with phenylalanine. As for the position corresponding to position 201 or 222 of SEQ ID NO: 2, for example, positions 201 and 222 in SEQ ID NO: 4, positions 201 and 222 in SEQ ID NO: 6, and positions 203 and 224 in SEQ ID NO: 8 correspond to these positions.

Thus, the parent polypeptide preferably has a tyrosine residue at position 201 or 222 of the amino acid sequence represented by SEQ ID NO: 4 or a position corresponding to the position 201 or 222. The polypeptide B) of the present invention is more preferably a mutant polypeptide in which tyrosine at the position 201 or 222 or the position corresponding to the position 201 or 222 is substituted with phenylalanine.

The parent polypeptide preferably has a tyrosine residue at position 201 or 222 of the amino acid sequence represented by SEQ ID NO: 6 or a position corresponding to the position 201 or 222. The polypeptide B) of the present invention is more preferably a mutant polypeptide in which tyrosine at the position 201 or 222 or the position corresponding to the position 201 or 222 is substituted with phenylalanine.

The parent polypeptide preferably has a tyrosine residue at position 203 or 224 of the amino acid sequence represented by SEQ ID NO: 8 or a position corresponding to the position 203 or 224. The polypeptide B) of the present invention is more preferably a mutant polypeptide in which tyrosine at the position 203 or 224 or the position corresponding to the position 203 or 224 is substituted with phenylalanine.

<Parent Polypeptide of Polypeptide C)>

In the polypeptide C), examples of the polypeptide consisting of an amino acid sequence having at least 90% identity to the amino acid sequence represented by SEQ ID NO: 2, and having 4-aminobenzoic acid hydroxylation activity include a polypeptide which consists of an amino acid sequence having at least 90% identity, specifically, 90% or higher, preferably 95% or higher, more preferably 96% or higher, further preferably 97 or higher, further preferably 98% or higher, further preferably 99% or higher identity to the amino acid sequence represented by SEQ ID NO: 2, and has 4-aminobenzoic acid hydroxylation activity.

The parent polypeptide preferably has a valine residue at position 47 of the amino acid sequence represented by SEQ ID NO: 2 or a position corresponding thereto, preferably has a histidine residue at position 72 thereof or a position corresponding thereto, preferably has a leucine residue at position 210 thereof or a position corresponding thereto, preferably has a threonine residue at position 294 thereof or a position corresponding thereto, or preferably has a tyrosine residue at position 385 thereof or a position corresponding thereto. For the polypeptide C) of the present invention, it is more preferred to substitute valine at the position 47 or the position corresponding thereto with isoleucine, serine, threonine, cysteine, methionine or glutamine, to substitute histidine at the position 72 or the position corresponding thereto with alanine or methionine, to substitute leucine at the position 210 or the position corresponding thereto with methionine, to substitute threonine at the position 294 or the position corresponding thereto with alanine, glycine, cysteine or serine, or to substitute tyrosine at the position 385 or the position corresponding thereto with valine, leucine, isoleucine or methionine, it is more preferred to substitute threonine at the position 294 or the position corresponding thereto with serine, to substitute valine at the position 47 or the position corresponding thereto with isoleucine, threonine, methionine or glutamine, or to substitute histidine at the position 72 or the position corresponding thereto with methionine, and it is further preferred to substitute threonine at the position 294 or the position corresponding thereto with serine or to substitute valine at the position 47 or the position corresponding thereto with isoleucine.

<Polynucleotide Encoding Polypeptide of Present Invention>

In the present invention, any of various mutagenesis techniques known in the art can be used as an approach of mutating the amino acid residue of the parent polypeptide. For example, a nucleotide sequence encoding the amino acid residue to be mutated in a polynucleotide encoding the amino acid sequence of the parent polypeptide (hereinafter, also referred to as a parent gene) can be mutated to a nucleotide sequence encoding a mutated amino acid residue to obtain a polynucleotide encoding the polypeptide of the present invention.

The introduction of the mutation of interest to the parent gene can be basically performed by use of any of various site-directed mutagenesis methods well known to those skilled in the art. The site-directed mutagenesis method can be performed by, for example, any approach such as inverse PCR or annealing. A commercially available kit for site-directed mutagenesis (e.g., QuikChange II Site-Directed Mutagenesis Kit or QuikChange Multi Site-Directed Mutagenesis Kit from Agilent Technologies, Inc.) may be used.

The site-directed mutagenesis of the parent gene can be performed most generally using primers for mutations containing a nucleotide mutation to be introduced. The primers for mutations can be designed so as to contain a nucleotide sequence which is annealed to a region containing a nucleotide sequence encoding the amino acid residue to be mutated in the parent gene, and has a nucleotide sequence (codon) encoding the mutated amino acid residue instead of the nucleotide sequence (codon) encoding the amino acid residue to be mutated. The nucleotide sequences (codons) encoding the unmutated or mutated amino acid residues can be appropriately recognized and selected by those skilled in the art on the basis of a usual textbook or the like. Alternatively, the site-directed mutagenesis may employ a method of linking DNA fragments respectively amplified from the upstream and downstream sides of a mutation site by separately using two complementary primers containing a nucleotide mutation to be introduced, into one fragment by SOE (splicing by overlap extension)-PCR (Gene, 1989, 77 (1): p. 61-68).

Template DNA containing the parent gene can be prepared by extracting genomic DNA by a routine method from a microorganism producing the 4-hydroxybenzoic acid hydroxylase mentioned above, or by extracting RNA therefrom and synthesizing cDNA by reverse transcription. Alternatively, a corresponding nucleotide sequence may be chemically synthesized on the basis of the amino acid sequence of the parent polypeptide, and used as template DNA. DNA sequences containing nucleotide sequences encoding HFM122, HFM388, HFM339, HFM77, HFM737, and HFMss0-1 already mentioned as polypeptides having 4-aminobenzoic acid hydroxylation activity are shown in SEQ ID NO: 1, SEQ ID NO: 3, SEQ ID NO: 5, SEQ ID NO: 7, SEQ ID NO: 9 and SEQ ID NO: 11, respectively.

The primers for mutations can be prepared by a well-known oligonucleotide synthesis method such as phosphoramidite method (Nucleic Acids Research, 1989, 17: 7059-7071). Such primer synthesis may be carried out using, for example, a commercially available oligonucleotide synthesis apparatus (manufactured by Applied Biosystems Inc. (ABI), etc.). The polynucleotide encoding the polypeptide of the present invention having the mutation of interest can be obtained by the site-directed mutagenesis as described above with the parent gene as template DNA using a primer set containing the primers for mutations.

The polynucleotide encoding the polypeptide of the present invention may contain single-stranded or double-stranded DNA, cDNA, RNA or other artificial nucleic acids. The DNA, the cDNA and the RNA may be chemically synthesized. The polynucleotide may also contain an open reading frame (ORF) as well as the nucleotide sequence of an untranslated region (UTR). The polynucleotide may be codon-optimized for the species of the transformant for mutant polypeptide production of the present invention. Information on codons which are used by various organisms is available from Codon Usage Database ([www.kazusa.or.jp/codon/]).

<Vector or DNA Fragment>

The obtained polynucleotide encoding the polypeptide of the present invention can be inserted into a vector. The vector containing the polynucleotide is an expression vector. Preferably, the vector is an expression vector which can introduce the polynucleotide encoding the polypeptide of the present invention into a host microorganism and enables the polynucleotide to be expressed within the host microorganism. Preferably, the vector contains the polynucleotide encoding the polypeptide of the present invention, and a control region operably linked thereto. The vector may be a vector capable of proliferating and replicating autonomously outside the chromosome (i.e., in a plasmid), or may be a vector which is integrated into the chromosome.

Specific examples of the vector include pBluescript II SK(−) (Stratagene California), pUC series vectors such as pUC18/19 and pUC118/119 (Takara Bio Inc.), pET series vectors (Takara Bio Inc.), pGEX series vectors (GE Healthcare Japan Corp.), pCold series vectors (Takara Bio Inc.), pHY300PLK (Takara Bio Inc.), pUB110 (Mckenzie, T. et al., 1986, Plasmid 15 (2): 93-103), pBR322 (Takara Bio Inc.), pRS403 (Stratagene California), pMW218/219 (Nippon Gene Co., Ltd.), pRI series vectors such as pRI909/910 (Takara Bio Inc.), pBI series vectors (Clontech Laboratories, Inc.), IN3 series vectors (Inplanta Innovations Inc.), pPTR1/2 (Takara Bio Inc.), pDJB2 (D. J. Ballance et al., Gene, 36, 321-331, 1985), pAB4-1 (van Hartingsveldt W et al., Mol Gen Genet, 206, 71-75, 1987), pLeu4 (M. I. G. Roncero et al., Gene, 84, 335-343, 1989), pPyr225 (C. D. Skory et al., Mol Genet Genomics, 268, 397-406, 2002), and pFG1 (Gruber, F. et al., Curr Genet, 18, 447-451, 1990).

The polynucleotide encoding the polypeptide of the present invention may be constructed as a DNA fragment containing the same. Examples of the DNA fragment include PCR-amplified DNA fragments and restriction enzyme-cleaved DNA fragments. Preferably, the DNA fragment can be an expression cassette containing the polynucleotide encoding the polypeptide of the present invention, and a control region operably linked thereto.

The control region contained in the vector or the DNA fragment is a sequence for allowing the polynucleotide encoding the polypeptide of the present invention to be expressed within a host cell into which the vector or the DNA fragment has been introduced. Examples thereof include expression regulation regions such as promoters and terminators, and replication origins. The type of the control region can be appropriately selected according to the type of the host microorganism into which the vector or the DNA fragment has been introduced. If necessary, the vector or the DNA fragment may further have a selective marker such as an antibiotic resistance gene or an amino acid synthesis-related gene (e.g., a resistance gene for a drug such as ampicillin, neomycin, kanamycin, or chloramphenicol).

The vector or the DNA fragment may contain a polynucleotide sequence encoding a polypeptide necessary for biosynthesizing 4-aminobenzoic acids. Examples of the polypeptide necessary for biosynthesizing 4-aminobenzoic acids include 4-amino-4-deoxychorismate synthase (pabAB) and 4-amino-4-deoxychorismate lyase (pabC).

The linkage of the polynucleotide encoding the polypeptide of the present invention to the control region or the marker gene sequence can be performed by a method such as the SOE-PCR mentioned above. Procedures of introducing the gene sequence into the vector are well known in the art. The type of the control region such as a promoter region, a terminator, or a secretion signal region is not particularly limited, and a promoter or a secretion signal sequence usually used can be appropriately selected and used according to the recipient host.

Preferred examples of the control region include, but are not particularly limited to, strong control regions which can enhance expression as compared with a wild type, for example, high-expression promoters known in the art such as T7 promoter, lac promoter, tac promoter, and trp promoter.

<Transformed Cell>

The transformed cell of the present invention can be obtained by introducing the vector comprising the polynucleotide encoding the polypeptide of the present invention into a host, or by introducing the DNA fragment containing the polynucleotide encoding the polypeptide of the present invention into the genome of a host.

The transformed cell is a cell expressibly harboring the polynucleotide encoding the polypeptide of the present invention, and can be a cell having the enhanced expression of the polynucleotide, and by extension, a cell having the enhanced expression of the polypeptide of the present invention.

Any of cells of a fungi, a yeast, actinomycete, E. coli, Bacillus subtilis, or the like may be used as a host cell, and E. coli or actinomycete is preferred. Examples of the actinomycete include bacteria of the genus Corynebacterium, bacteria of the genus Mycobacterium, bacteria of the genus Rhodococcus, bacteria of the genus Streptomyces, and bacteria of the genus Propionibacterium. A bacterium of the genus Corynebacterium is preferred, and Corynebacterium glutamicum is more preferred.

Among others, a microorganism which can supply 4-aminobenzoic acids serving as substrates in the biosynthesis of 4-amino-3-hydroxybenzoic acids is preferred, and a microorganism having enhanced ability to supply 4-aminobenzoic acids is more preferred. Examples of the method for enhancing the ability of the microorganism to supply 4-aminobenzoic acids include a method of introducing, into the microorganism, a vector containing a polynucleotide encoding a polypeptide necessary for biosynthesizing 4-aminobenzoic acids, and a control region operably linked thereto, and a method of substituting the control region of a polynucleotide encoding a polypeptide necessary for biosynthesizing 4-aminobenzoic acids, which is originally carried by the microorganism, with a strong-expression promoter.

Examples of the method for introducing the vector or the DNA fragment into the host include, for example, electroporation, transformation, transfection, conjugation, protoplast method, particle gun method, or Agrobacterium method.

Examples of the method for introducing the polynucleotide into the genome of the host include, but are not particularly limited to, a double crossover method using the DNA fragment containing the polynucleotide. The DNA fragment may be introduced to downstream of a promoter sequence of a gene having a large expression level in the host cell mentioned above, or a fragment of the DNA fragment operably linked to the control region mentioned above may be prepared in advance, and the linked fragment can be introduced into the genome of the host. The DNA fragment may be further linked in advance to a marker (drug resistance gene, auxotrophic complementary gene, etc.) for selecting a cell properly harboring the polynucleotide of the present invention.

The transformed cell harboring the vector or the DNA fragment of interest can be selected by using the selective marker. When the selective marker is, for example, an antibiotic resistance gene, the transformed cell into which the vector or the DNA fragment of interest has been introduced can be selected by culture in a culture medium supplemented with the antibiotic. When the selective marker is, for example, an amino acid synthesis-related gene, the transformed cell harboring the vector or the DNA fragment of interest can be selected by using the presence or absence of the amino acid auxotrophy as an index after introduction of gene into a host microorganism requiring the amino acid. Alternatively, the introduction of the vector or the DNA fragment of interest may be confirmed by examining the DNA sequence of the transformed cell by PCR or the like.

The transformed cell obtained above is cultured in a proper culture medium so that the polynucleotide introduced into the cell is expressed to produce the polypeptide of the present invention. Specifically, the transformed cell is capable of serving as a bacterium producing a polypeptide having 4-aminobenzoic acid hydroxylation activity. In the case of culturing the transformed cell of the present invention, as shown in Examples mentioned later, the productivity of 4-amino-3-hydroxybenzoic acid is improved as compared with the case of using a transformed cell producing the parent polypeptide.

Specifically, in a polypeptide consisting of the amino acid sequence represented by SEQ ID NO: 2 or an amino acid sequence having at least 47% identity thereto, and having 4-aminobenzoic acid hydroxylation activity, the mutation to substitute an amino acid residue at position 47 of the amino acid sequence represented by SEQ ID NO: 2 or a position corresponding thereto with leucine is useful in improvement in 4-aminobenzoic acid hydroxylation activity and also useful in improvement in the productivity of 4-amino-3-hydroxybenzoic acids. In a polypeptide consisting of the amino acid sequence represented by SEQ ID NO: 2 or an amino acid sequence having at least 51′% identity thereto, and having 4-aminobenzoic acid hydroxylation activity, the mutation to substitute an amino acid residue at position 201 or 222 of the amino acid sequence represented by SEQ ID NO: 2 or a position corresponding to the position 201 or 222 with phenylalanine is useful in improvement in 4-aminobenzoic acid hydroxylation activity and also useful in improvement in the productivity of 4-amino-3-hydroxybenzoic acids.

In a polypeptide consisting of the amino acid sequence represented by SEQ ID NO: 2 or an amino acid sequence having at least 90% identity thereto, and having 4-aminobenzoic acid hydroxylation activity, the mutation to substitute an amino acid residue at position 47, 72, 210, 294 or 385 of the amino acid sequence represented by SEQ ID NO: 2 or a position corresponding to the position 47, 72, 210, 294 or 385 with the following amino acid is useful in improvement in 4-aminobenzoic acid hydroxylation activity and also useful in improvement in the productivity of 4-amino-3-hydroxybenzoic acids:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

Furthermore, the transformed cell of the present invention serves as a bacterium producing a polypeptide and having improved 4-aminobenzoic acid hydroxylation activity and serves as a useful strain producing 4-amino-3-hydroxybenzoic acids.

<Production of 4-Amino-3-Hydroxybenzoic Acids>

The method for producing 4-amino-3-hydroxybenzoic acids according to the present invention includes a step of culturing the transformed cell of the present invention, and 4-amino-3-hydroxybenzoic acids can be obtained by collecting the 4-amino-3-hydroxybenzoic acids from a culture medium.

In the present invention, specific examples of the 4-amino-3-hydroxybenzoic acids include 4-amino-3-hydroxybenzoic acid derivatives of the following formula (1):

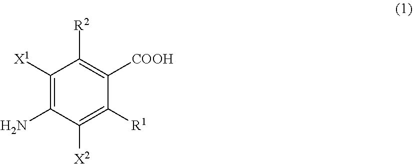

wherein R¹ represents a hydrogen atom, a hydroxy group (—OH), a methoxy group (—OCH₃), an amino group (—NH₂), a fluorine atom (—F), a chlorine atom (—Cl), a bromine atom (—Br), an iodine atom (—I), a carboxy group (—COOH), a methyl group (—CH₃), or an ethyl group (—CH₂CH₃), R² represents a hydrogen atom, a hydroxy group (—OH), a methoxy group (—OCH₃), an amino group (—NH₂), a fluorine atom (—F), a chlorine atom (—Cl), a bromine atom (—Br), an iodine atom (—I), a carboxy group (—COOH), a methyl group (—CH₃) or an ethyl group (—CH₂CH₃), and X¹ and X² each represent a hydrogen atom or a hydroxy group, at least one of which represents a hydroxy group.

The functional group R¹ is preferably a hydrogen atom, a hydroxy group (—OH), a methoxy group (—OCH₃), a fluorine atom (—F) or a methyl group (—CH₃).

The functional group R² is preferably a hydrogen atom, a hydroxy group (—OH), a methoxy group (—OCH₃), a fluorine atom (—F) or a methyl group (—CH₃).

More preferably, both R¹ and R² are hydrogen atoms.

Both X¹ and X² may be hydroxy groups, and either one of X¹ or X² is preferably a hydroxy group.

The culture medium can contain, if necessary, 4-aminobenzoic acids serving as substrates in the biosynthesis of 4-amino-3-hydroxybenzoic acids.

In this context, examples of the 4-aminobenzoic acids include 4-aminobenzoic acid derivatives of the following formula (2):

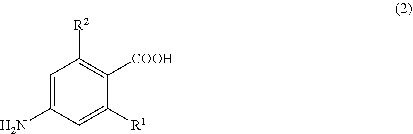

wherein R¹ and R² are as defined above.

Any of a natural culture medium and a synthetic culture medium may be used as the culture medium for the culture of the transformed cell as long as the culture medium contains a carbon source, a nitrogen source, inorganic salts, etc. and permits efficient culture of the transformed cell of the present invention. For example, saccharides such as glucose, polyols such as glycerin, alcohols such as ethanol, or organic acids such as pyruvic acid, succinic acid or citric acid can be used as carbon sources. For example, peptone, meat extracts, yeast extracts, casein hydrolysates, alkaline extracts of soybean meal, alkylamines such as methylamine, or ammonia or its salt can be used as nitrogen sources. In addition, phosphate, carbonate, sulfate, salts of magnesium, calcium, potassium, iron, manganese, zinc, or the like, a particular amino acid, a particular vitamin, an antifoaming agent, etc. may be used, if necessary.

The culture can usually be performed, if necessary, with stirring or shaking, at 10° C. to 40° C. for 6 hours to 72 hours, preferably for 9 hours to 60 hours, more preferably for 12 hours to 48 hours. During the culture, an antibiotic such as ampicillin or kanamycin may be added, if necessary, to the culture medium.

The methods for collecting and purifying 4-amino-3-hydroxybenzoic acids from the cultures are not particularly limited. Specifically, the collection and the purification can be carried out by combining a well-known ion-exchange resin method, precipitation method, crystallization method, recrystallization method, concentration method and other methods. For example, after removal of bacterial cells by centrifugation or the like, ionic substances are removed with cation- and anion-exchange resins, and the resultant can be concentrated to obtain 4-amino-3-hydroxybenzoic acids. The 4-amino-3-hydroxybenzoic acids accumulated in the cultures may be used directly without being isolated.

The present invention also encompasses the following substances, production methods, use, methods, etc. as exemplary embodiments. However, the present invention is not limited by these embodiments.

<1> A polypeptide having 4-aminobenzoic acid hydroxylation activity, the polypeptide being selected from the group consisting of the following A) to C):

A) a polypeptide having 4-aminobenzoic acid hydroxylation activity, consisting of the amino acid sequence represented by SEQ ID NO: 2 or an amino acid sequence having at least 47′% identity thereto, and having an amino acid residue at position 47 of the amino acid sequence represented by SEQ ID NO: 2 or a position corresponding thereto being leucine;

B) a polypeptide having 4-aminobenzoic acid hydroxylation activity, consisting of the amino acid sequence represented by SEQ ID NO: 2 or an amino acid sequence having at least 51′% identity thereto, and having an amino acid residue at position 201 or 222 of the amino acid sequence represented by SEQ ID NO: 2, or a position corresponding to the position 201 or 222 being phenylalanine; and

C) a polypeptide having 4-aminobenzoic acid hydroxylation activity, consisting of the amino acid sequence represented by SEQ ID NO: 2 or an amino acid sequence having at least 905 identity thereto, and having an amino acid residue at position 47, 72, 210, 294 or 385 of the amino acid sequence represented by SEQ ID NO: 2, or a position corresponding to the position 47, 72, 210, 294 or 385 is the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

<2> A polypeptide having 4-aminobenzoic acid hydroxylation activity, the polypeptide being selected from the group consisting of the following A″) to C″):

A″) a mutant polypeptide having 4-aminobenzoic acid hydroxylation activity, consisting of the amino acid sequence represented by SEQ ID NO: 2 or an amino acid sequence having at least 47% identity thereto, wherein an amino acid residue at position 47 of the amino acid sequence represented by SEQ ID NO: 2 or a position corresponding thereto is substituted with leucine;

B″) a polypeptide having 4-aminobenzoic acid hydroxylation activity, consisting of the amino acid sequence represented by SEQ ID NO: 2 or an amino acid sequence having at least 51% identity thereto, an amino acid residue at position 201 or 222 of the amino acid sequence represented by SEQ ID NO: 2, or a position corresponding to the position 201 or 222 is substituted with phenylalanine; and

C″) a polypeptide having 4-aminobenzoic acid hydroxylation activity, consisting of the amino acid sequence represented by SEQ ID NO: 2 or an amino acid sequence having at least 90% identity thereto, an amino acid residue at position 47, 72, 210, 294 or 385 of the amino acid sequence represented by SEQ ID NO: 2, or a position corresponding to the position 47, 72, 210, 294 or 385 is substituted with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

<3> The mutant polypeptide according to <2>, wherein the substitution of an amino acid residue in A″) is substitution of valine with leucine, the substitution an amino acid residue in B″) is substitution of tyrosine with phenylalanine, and the substitution of an amino acid residue in C″) is substitution of threonine at position 294 or a position corresponding thereto with serine, substitution of valine at position 47 or a position corresponding thereto with isoleucine, threonine, methionine or glutamine, or substitution of histidine at position 72 or a position corresponding thereto with methionine.

<4> A method for producing a mutant polypeptide having 4-aminobenzoic acid hydroxylation activity, comprising substituting an amino acid residue, the substituting is selected from the group consisting of the following A′) to C′):

A′) in a polypeptide consisting of the amino acid sequence represented by SEQ ID NO: 2 or an amino acid sequence having at least 47′% identity thereto, and having 4-aminobenzoic acid hydroxylation activity, substitution of an amino acid residue at position 47 of the amino acid sequence represented by SEQ ID NO: 2 or a position corresponding thereto with leucine;

B′) in a polypeptide consisting of the amino acid sequence represented by SEQ ID NO: 2 or an amino acid sequence having at least 51, identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and

C′) in a polypeptide consisting of the amino acid sequence represented by SEQ ID NO: 2 or an amino acid sequence having at least 90, identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

<5> The method according to <4>, wherein the substitution of an amino acid residue in A′) is substitution of valine with leucine, the substitution of an amino acid residue in B′) is substitution of tyrosine with phenylalanine, and the substitution of an amino acid residue in C′) is substitution of threonine at position 294 or a position corresponding thereto with serine, substitution of valine at position 47 or a position corresponding thereto with isoleucine, threonine, methionine or glutamine, or substitution of histidine at position 72 or a position corresponding thereto with methionine.

<6> A method for improving 4-aminobenzoic acid hydroxylation activity, comprising substituting an amino acid residue, the substituting is selected from the group consisting of the following A′) to C′):

A′) in a polypeptide consisting of the amino acid sequence represented by SEQ ID NO: 2 or an amino acid sequence having at least 47, identity thereto, and having 4-aminobenzoic acid hydroxylation activity, substitution of an amino acid residue at position 47 of the amino acid sequence represented by SEQ ID NO: 2 or a position corresponding thereto with leucine;

B′) in a polypeptide consisting of the amino acid sequence represented by SEQ ID NO: 2 or an amino acid sequence having at least 51% identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and

C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

<7> The method according to <6>, wherein the substitution of amino acid residue in A′) is substitution of valine with leucine, the substitution of an amino acid residue in B′) is substitution of tyrosine with phenylalanine, and the substitution of an amino acid residue in C′) is substitution of threonine at position 294 or a position corresponding thereto with serine, substitution of valine at position 47 or a position corresponding thereto with isoleucine, threonine, methionine or glutamine, or substitution of histidine at position 72 or a position corresponding thereto with methionine.

<8> A method for improving the productivity of 4-aminobenzoic acids, comprising substituting an amino acid residue, the substituting is selected from the group consisting of the following A′) to C′):

A′) in the case of producing 4-amino-3-hydroxybenzoic acids using a polypeptide consisting of the amino acid sequence represented by SEQ ID NO: 2 or an amino acid sequence having at least 47% identity thereto, and having 4-aminobenzoic acid hydroxylation activity, substitution of an amino acid residue at position 47 of the amino acid sequence represented by SEQ ID NO: 2 or a position corresponding thereto with leucine;

B′) in a polypeptide consisting of the amino acid sequence represented by SEQ ID NO: 2 or an amino acid sequence having at least 515 identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and

C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:

(a) the position 47 or the position corresponding thereto: isoleucine, serine, threonine, cysteine, methionine, or glutamine,

(b) the position 72 or the position corresponding thereto: alanine or methionine,

(c) the position 210 or the position corresponding thereto: methionine,

(d) the position 294 or the position corresponding thereto: alanine, glycine, cysteine, or serine, and

(e) the position 385 or the position corresponding thereto: valine, leucine, isoleucine, or methionine.

<9> The method according to <8>, wherein the substitution of an amino acid residue in A′) is substitution of valine with leucine, the substitution of an amino acid residue in B′) is substitution of tyrosine with phenylalanine, and the substitution of an amino acid residue in C′) is substitution of threonine at position 294 or a position corresponding thereto with serine, substitution of valine at position 47 or a position corresponding thereto with isoleucine, threonine, methionine or glutamine, or substitution of histidine at position 72 or a position corresponding thereto with methionine.

<10> A polynucleotide encoding the polypeptide according to any of <1> to <3>.

<11> A vector or a DNA fragment comprising the polynucleotide according to <10>.

<12> A transformed cell comprising the vector or the DNA fragment according to <11>.

<13> The transformed cell according to <12>, wherein the transformed cell is derived from E. coli or a bacterium of the genus Corynebacterium.

<14> The transformed cell according to <12> or <13>, wherein the transformed cell is a microorganism capable of supplying 4-aminobenzoic acids.

<15> The transformed cell according to <12> or <13>, wherein the transformed cell has improved ability to supply 4-aminobenzoic acids.

<16> A method for producing 4-amino-3-hydroxybenzoic acids, comprising a step of culturing the transformed cell according to any of <12> to <15>.

<17> The method according to <16>, wherein the culturing is performed in a culture medium containing a saccharide as a carbon source.

<18> The method according to <16> or <17>, further comprising a step of collecting 4-amino-3-hydroxybenzoic acids from a culture medium.

<19> The method according to any of <16> to <18>, wherein the culture is performed in the presence of 4-aminobenzoic acids.

<20> The method according to any of <16> to <19>, wherein the 4-amino-3-hydroxybenzoic acids are 4-amino-3-hydroxybenzoic acid derivatives of the following formula (1):

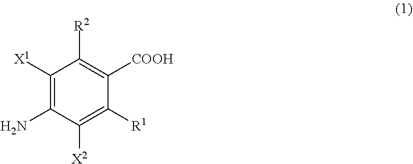

wherein R¹ represents a hydrogen atom, a hydroxy group, a methoxy group, an amino group, a fluorine atom, a chlorine atom, a bromine atom, an iodine atom, a carboxy group, a methyl group, or an ethyl group, R² represents a hydrogen atom, a hydroxy group, a methoxy group, an amino group, a fluorine atom, a chlorine atom, a bromine atom, an iodine atom, a carboxy group, a methyl group, or an ethyl group, and X¹ and X² each represent a hydrogen atom or a hydroxy group, at least one of which represents a hydroxy group.

<21> The method according to <19> or <20>, wherein the 4-aminobenzoic acids are 4-aminobenzoic acid derivatives of the following formula (2):

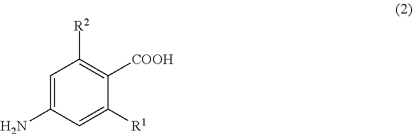

wherein R¹ represents a hydrogen atom, a hydroxy group, a methoxy group, an amino group, a fluorine atom, a chlorine atom, a bromine atom, an iodine atom, a carboxy group, a methyl group, or an ethyl group, and R² represents a hydrogen atom, a hydroxy group, a methoxy group, an amino group, a fluorine atom, a chlorine atom, a bromine atom, an iodine atom, a carboxy group, a methyl group, or an ethyl group.

### EXAMPLES

Hereinafter, the present invention will be described in more detail with reference to Examples. However, the present invention is not limited thereby.

### Example A1 Production of 4-Amino-3-Hydroxybenzoic Acid

In the following Example, PCR was performed using PrimeSTAR Max Premix (Takara Bio Inc.).

(1) Preparation of Plasmid Containing Gene Encoding Wild-Type Enzyme

(a) Preparation of Plasmid pECsf_gapS_pabABC

A DNA fragment containing genes encoding 4-amino-4-deoxychorismate synthase and 4-amino-4-deoxychorismate lyase was amplified by PCR using the genome extracted from the Corynebacterium glutamicum ATCC13032 strain by a routine method as a template and using primers GN14_127 (SEQ ID NO: 13, TATTAATTAAATGCGCGTTTTAATTATTGATAATTATGATTC) and GN14_133 (SEQ ID NO: 14, TTGCGGCCGCTTGTTTAAACCTCCTTACAGAAAAATGGTTGGGCG). This fragment was inserted between the PacI site and the NotI site of a plasmid pECsf_gapS (see Japanese Patent Application No. 2015-25491) to obtain a plasmid pECsf_gapS_pabABC.

(b) Preparation of Plasmid pECsf_gapS_pabABC_HFM122

A DNA fragment for a vector was synthesized by PCR using the plasmid pECsf_gapS_pabABC obtained above as a template and using primers pabABCcory vec R (SEQ ID NO: 15, AAATTTAAACCTCCTTTACAGAAAAATGGTTGG) and pabABCcory vec F (SEQ ID NO: 16, GGAGGTTTAAACAAGCGGCCGCGATATC). Subsequently, a plasmid containing a gene (SEQ ID NO: 1) encoding a polypeptide HFM122 having 4-aminobenzoic acid hydroxylation activity was prepared by artificial gene synthesis, and a DNA fragment for an insert was synthesized by PCR using this plasmid as a template and using primers pECsfD HFM122 F (SEQ ID NO: 17, AGGAGGTTTAAATTTATGCGCACTCAGGTGGCTAT) and pECsfD HFM122 R (SEQ ID NO: 18, CTTGTTTAAACCTCCTTATACGAGTGGCAGTCCTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_H4FM122. The ECOS Competent E. coli DH5a strain (Nippon Gene Co., Ltd.) was transformed with the obtained plasmid solution. The cell solution was spread over LBKm agar medium (1% Bacto Tryptone, 0.5% yeast extract, 1% NaCl, 50 μg/mL kanamycin sulfate, 1.5% agar) and then left standing overnight at 37° C. The obtained colonies were subjected to PCR reaction using Sapphire Amp (Takara Bio Inc.) and primers pabABC+pobA for CPCR F (SEQ ID NO: 19, GCTATCAAAACATTCGGCACATTGGTTTTCC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium (1% Bacto Tryptone, 0.5% yeast extract, 1% NaCl, 50 μg/mL kanamycin sulfate) and cultured overnight at 37° C. A plasmid was purified from this culture solution using NucleoSpin Plasmid EasyPure (Takara Bio Inc.).

(c) Preparation of Plasmid

pECsf_gapS_pabABC_tuD_HFM122

A DNA fragment for a vector was synthesized by PCR using the plasmid pECsf_gapS_pabABC_HFM122 obtained above as a template and using primers pabC last R (SEQ ID NO: 21, TTACAGAAAAATGGTTGGGCGCAA) and HFM122 F (SEQ ID NO: 22, ATGCGCACTCAGGTGGCTATCG). Subsequently, a DNA fragment (SEQ ID NO: 23, TACGTACCTGCAGGTAGCGTGTCAGTAGGCGCGTAGGGTAAGTGGGGTAGCGGCTTG TTAGATATCTTGAAATCGGCTTTCAACAGCATTGATTTCGATGTATTTAGCTGGCCG TTACCCTGCGAATGTCCACAGGGTAGCTGGTAGTTTGAAAATCAACGCCGTTGCCCT TAGGATTCAGTAACTGGCACATTTTGTAATGCGCTAGATCTGTGTGCTCAGTCTTCC AGGCTGCTTATCACAGTGAAAGCAAAACCAATTCGTGGCTGCGAAAGTCGTAGCCAC CACGAAGTCCAAAGGAGGATCTAAATTATGAATAATATAAAAGGAGGAATTAATTAA) containing tuf gene (cg0587) promoter (hereinafter, referred to as tu promoter) carried by the Corynebacterium glutamicum ATCC13032 strain was prepared by artificial gene synthesis, and a DNA fragment for an insert was synthesized by PCR using this fragment as a template and using primers pabC-Ptu F (SEQ ID NO: 24, ACCATTTTTCTGTAATACGTACCTGCAGGTAGCGTG) and Ptu-HFM122 R (SEQ ID NO: 25, CACCTGAGTGCGCATTTAATTAATTCCTCCTTTTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tuD_HFM122. The ECOS Competent E. coli DH5a strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 26, GCTTGTTAGATATCTTGAAATCGGCTTTC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmid, the genes encoding 4-amino-4-deoxychorismate synthase and 4-amino-4-deoxychorismate lyase were linked under the control of gap promoter, and the gene encoding wild-type HFM122 was further linked under the control of the tu promoter.

(d) Preparation of Other Plasmids

A DNA fragment for a vector was synthesized by PCR using the plasmid pECsf_gapS_pabABC_tuD_HFM122 obtained above as a template and using primers pGapABA_tu vec F (SEQ ID NO: 27, GGAGGTTTAAACAAGCGG) and pGapABA_tu vec R (SEQ ID NO: 28, AATTTAGATCCTCCTTTGGACTTCGTG). Subsequently, a plasmid containing a gene (SEQ ID NO: 3, 5, 7, 9, or 11) encoding each polypeptide having 4-aminobenzoic acid hydroxylation activity was prepared by artificial gene synthesis, and a DNA fragment for an insert was synthesized by PCR using this plasmid as a template and using primers shown in the column “Primer” of Table A1. These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid shown in the column “Plasmid” of Table 1. The ECOS Competent E. coli DH5α strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 26, GCTTGTTAGATATCTTGAAATCGGCTTTC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmids, the genes encoding 4-amino-4-deoxychorismate synthase and 4-amino-4-deoxychorismate lyase were linked under the control of gap promoter, and the gene encoding wild-type hydroxylase was further linked under the control of the tu promoter.

TABLE A1

| | | | SEQ |
|---|---|---|---|
| | | | ID |
| Plasmid | Primer | Sequence (5′-→3′) | NO |
| pECsf_ | HFM77 | AGGAGGATCTAAATTATGCGT | 29 |
| gapS_ | ins F | ACTCAGGTGGGAATC | |
| pabAB | HFM77 | CTTGTTTAAACCTCCTTAAGC | 30 |
| C_tu_ | ins R | AAGTGGCATGG | |
| HFM77 | | | |
| pECsf_ | HFM339 | AGGAGGATCTAAATTATGCGC | 31 |
| gapS_ | ins F | ACTCAGGTGGCAATC | |
| pabAB | HFM339 | CTTGTTTAAACCTCCTTAGTA | 32 |
| C_tu_ | ins R | TGGCAGGCCTACG | |
| HFM339 | | | |
| pECsf_ | HFM388 | AGGAGGATCTAAATTATGCGC | 33 |
| gapS_ | ins F | ACCCAAGTGGTCATC | |
| pabAB | HFM388 | CTTGTTTAAACCTCCTTAGAA | 34 |
| C_tu_ | ins R | CGGCAGACCCACGTAG | |
| HFM388 | | | |
| pECsf_ | HFM737 | AGGAGGATCTAAATTATGCGC | 35 |
| gapS_ | ins F | ACTCAGGTTGGTATC | |
| pabAB | HFM737 | CTTGTTTAAACCTCCTTAGTG | 36 |
| C_tu_ | ins R | GCTCAGTCCAACCATTC | |
| HFM737 | | | |
| pECsf_ | HFMss0-1 | AGGAGGATCTAAATTATGCGT | 37 |
| gapS_ | ins F | ACCCAAGTGGCCATCATTG | |
| pabAB | HFMss0-1 | CTTGTTTAAACCTCCTTAGAA | 38 |
| C_tu_ | ins R | GCCAATCGGAAGGCC | |
| HFMss0-1 | | | |

(2) Preparation of Plasmid Containing Gene Encoding Mutant Enzyme

The preparation of a plasmid containing a gene encoding a mutant enzyme will be given below by taking, as an example, the preparation of a plasmid containing a gene encoding a mutant enzyme in which valine at position 47 of HFM77 was substituted with leucine.

A plasmid pECsf_gapS_pabABC_tu_HFM77 V47L was constructed by PCR using a plasmid pECsf_gapS_pabABC_tu_HFM77 as a template and using complementary primers HFM77 V47L F (SEQ ID NO: 39, GCCGGGCTCCTGGAACAGTCTACGGTT) and HFM77 V47L R (SEQ ID NO: 40, TTCCAGGAGCCCGGCGCGGATGGTCTG). The PCR product was treated with DpnI (Takara Bio Inc.). The ECOS Competent E. coli DH5α strain (Nippon Gene Co., Ltd.) was transformed with the solution thus treated. The cell solution was spread over LBKm agar medium and then left standing overnight at 37° C. The obtained colonies were selected as a transformant. The transformant was inoculated to 2 mL of LBKm liquid medium and cultured overnight at 37° C. A plasmid was purified from this culture solution using NucleoSpin Plasmid EasyPure (Takara Bio Inc.).

In a similar manner, a plasmid containing a gene encoding each enzyme mutant was obtained by PCR using a plasmid shown in the column “Template” of Table A2 instead of the plasmid pECsf_gapS_pabABC_tu_HFM77 and using primers shown in the column “Primer” of Table A2 instead of the primers HFM77 V47L F and HFM77 V47L R.

TABLE A2

| | | | SEQ |
|---|---|---|---|
| | | | ID |
| Template | Primer | Sequence (5′-→3′) | NO |
| pECsf_gapS_ | HFM122 | GCTGGTCTCCTGGAACGT | 41 |
| pabABC_tuD_ | V47L F | ATCACGGTG | |
| HFM122 | HFM122 | TTCCAGGAGACCAGCCCG | 42 |
| | V47L R | AACTCGGCC | |
| pECsf_gapS_ | HFM339 | GCAGGCCTCCTGGAGCAG | 43 |
| pabABC_tu_ | V47L F | GGCATGGTT | |
| HFM339 | HFM339 | CTCCAGGAGGCCTGCACG | 44 |
| | V47L R | GATGCGGGA | |
| pECsf_gapS_ | HFM388 | GCTGGACTCTTGGAACAG | 45 |
| pabABC_tu_ | V47L F | GGCACCGTT | |
| HFM388 | HFM388 | TTCCAAGAGTCCAGCGCG | 46 |
| | V47L R | AACTCGCCC | |
| pECsf_gapS_ | HFM737 | GCGGGTCTCCTGGAACAG | 47 |
| pabABC_tu_ | V47L F | GGCACCATG | |
| HFM737 | HFM737 | TTCCAGGAGACCCGCCCG | 48 |
| | V47L R | AATCGTGGA | |
| pECs_gapS_ | HFM330-1 | GCCGGTCTCCTGGAGCAC | 49 |
| pabABC_tu_ | V47L F | TCCACGGTG | |
| HFMss0-1 | HFM330-1 | CTCCAGGAGACCGGCACG | 50 |
| | V47L R | GATGCGTGA | |

(3) Introduction of Plasmid into Host Cell

The Corynebacterium glutamicum DRHG145 strain (see Japanese Patent Application No. 2014-523757) was transformed with each plasmid obtained above by electroporation (Bio-Rad Laboratories, Inc.). The obtained transformed cell solution was spread over LBKm agar medium and then left standing at 30° C. for 2 days. The obtained colonies were used as a transformant.

(4) Culture of Transformant

Each transformant obtained above was inoculated to 1 mL of CGYE medium (containing 50 μg/mL kanamycin sulfate) shown in Table A3, and cultured overnight at 30° C. 100 μL of the obtained culture solution was inoculated to 10 mL of CGXII medium (containing 50 μg/mL kanamycin sulfate) shown in Table A4, and cultured at 30° C. for approximately 48 hours. Then, bacterial cells were removed by centrifugation to obtain a culture supernatant. The concentration of 4-amino-3-hydroxybenzoic acid in the obtained culture supernatant was quantified in accordance with the method of Reference Example 1. The rate of improvement in the ability to produce 4-amino-3-hydroxybenzoic acid was calculated according to the equation given below. In the equation, “WT” represents a “transformant harboring the plasmid containing the gene encoding the wild-type enzyme”, and “MT” represents a “transformant harboring the plasmid containing the gene encoding the mutant enzyme and prepared from the plasmid containing the gene encoding the wild-type enzyme has been introduced”.

Rate of improvement in production ability=Ability of MT to produce 4-amino-3-hydroxybenzoic acid/Ability of WT to produce 4-amino-3-hydroxybenzoic acid (Equation 1)

TABLE A3
CGYE medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Yeast extract | 1 | g |

TABLE A4
CGXII medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Tryptone | 10 | g |

(5) Results

As shown in Table A5, the bacterial strain harboring each mutant enzyme had the more improved ability to produce 4-amino-3-hydroxybenzoic acid than the bacterial strain harboring the wild-type enzyme.

TABLE A5

| | Ability to produce 4- | |
|---|---|---|
| | amino-3-hydroxybenzoic | Rate of improvement |
| Hydroxylase | acid (g/L) | in production ability |
| HFM77 wt | 0.073 | 1.00 |
| HFM77 V47L | 0.134 | 1.83 |
| HFM122 wt | 0.134 | 1.00 |
| HFM122 V47L | 0.372 | 2.77 |
| HFM339 wt | 0.016 | 1.00 |
| HFM339 V47L | 0.071 | 4.47 |
| HFM388 wt | 0.033 | 1.00 |
| HFM388 V47L | 0.100 | 3.02 |
| HFM737 wt | 0.112 | 1.00 |
| HFM737 V47L | 0.164 | 1.46 |
| HFMss0-1 wt | 0.112 | 1.00 |
| HFMss0-1 V47L | 0.140 | 1.25 |

### Example B1 Production of 4-Amino-3-Hydroxybenzoic Acid

In the following Example, PCR was performed using PrimeSTAR Max Premix (Takara Bio Inc.).

(1) Preparation of Plasmid Containing Gene Encoding Wild-Type Enzyme

(a) Preparation of Plasmid pECsf_gapS_pabABC

A DNA fragment containing genes encoding 4-amino-4-deoxychorismate synthase and 4-amino-4-deoxychorismate lyase was amplified by PCR using the genome extracted from the Corynebacterium glutamicum ATCC13032 strain by a routine method as a template and using primers GN14_127 (SEQ ID NO: 13, TATTAATTAAATGCGCGTTTTAATTATTGATAATTATGATTC) and GN14_133 (SEQ ID NO: 14, TTGCGGCCGCTTGTTTAAACCTCCTTACAGAAAAATGGTTGGGCG). This fragment was inserted between the PacI site and the NotI site of a plasmid pECsf_gapS (see Japanese Patent Application No. 2015-25491) to obtain a plasmid pECsf_gapS_pabABC.

(b) Preparation of Plasmid pECsf_gapS_pabABC_HFM122

A DNA fragment for a vector was synthesized by PCR using the plasmid pECsf_gapS_pabABC obtained above as a template and using primers pabABCcory vec R (SEQ ID NO: 15, AAATTTAAACCTCCTTTACAGAAAAATGGTTGG) and pabABCcory vec F (SEQ ID NO: 16, GGAGGTTTAAACAAGCGGCCGCGATATC). Subsequently, a plasmid containing a gene (SEQ ID NO: 1) encoding a polypeptide HFM122 having 4-aminobenzoic acid hydroxylation activity was prepared by artificial gene synthesis, and a DNA fragment for an insert was synthesized by PCR using this plasmid as a template and using primers pECsfD HFM122 F (SEQ ID NO: 17, AGGAGGTTTAAATTTATGCGCACTCAGGTGGCTAT) and pECsfD HFM122 R (SEQ ID NO: 18, CTTGTTTAAACCTCCTTATACGAGTGGCAGTCCTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_HFM122. The ECOS Competent E. coli DH5α strain (Nippon Gene Co., Ltd.) was transformed with the obtained plasmid solution. The cell solution was spread over LBKm agar medium (l Bacto Tryptone, 0.5% yeast extract, 1% NaCl, 50 μg/mL kanamycin sulfate, 1.5% agar) and then left standing overnight at 37° C. The obtained colonies were subjected to PCR reaction using Sapphire Amp (Takara Bio Inc.) and primers pabABC+pobA for CPCR F (SEQ ID NO: 19, GCTATCAAAACATTCGGCACATTGGTTTTCC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium (1 Bacto Tryptone, 0.5% yeast extract, 1% NaCl, 50 μg/mL kanamycin sulfate) and cultured overnight at 37° C. A plasmid was purified from this culture solution using NucleoSpin Plasmid EasyPure (Takara Bio Inc.).

(c) Preparation of Plasmid

pECsf_gapS_pabABC_tuD_HFM122

A DNA fragment for a vector was synthesized by PCR using the plasmid pECsf_gapS_pabABC_HFM122 obtained above as a template and using primers pabC last R (SEQ ID NO: 21, TTACAGAAAAATGGTTGGGCGCAA) and HFM122 F (SEQ ID NO: 22, ATGCGCACTCAGGTGGCTATCG). Subsequently, a DNA fragment (SEQ ID NO: 23, TACGTACCTGCAGGTAGCGTGTCAGTAGGCGCGTAGGGTAAGTGGGGTAGCGGCTTG TTAGATATCTTGAAATCGGCTTTCAACAGCATTGATTTCGATGTATTTAGCTGGCCG TTACCCTGCGAATGTCCACAGGGTAGCTGGTAGTTTGAAAATCAACGCCGTTGCCCT TAGGATTCAGTAACTGGCACATTTTGTAATGCGCTAGATCTGTGTGCTCAGTCTTCC AGGCTGCTTATCACAGTGAAAGCAAAACCAATTCGTGGCTGCGAAAGTCGTAGCCAC CACGAAGTCCAAAGGAGGATCTAAATTATGAATAATATAAAAGGAGGAATTAATTAA) containing tuf gene (cg0587) promoter (hereinafter, referred to as tu promoter) carried by the Corynebacterium glutamicum ATCC13032 strain was prepared by artificial gene synthesis, and a DNA fragment for an insert was synthesized by PCR using this fragment as a template and using primers pabC-Ptu F (SEQ ID NO: 24, ACCATTTTTCTGTAATACGTACCTGCAGGTAGCGTG) and Ptu-HFM122 R (SEQ ID NO: 25, CACCTGAGTGCGCATTTAATTAATTCCTCCTTTTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tuD_HFM122. The ECOS Competent E. coli DH5α strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 26, GCTTGTTAGATATCTTGAAATCGGCTTTC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmid, the genes encoding 4-amino-4-deoxychorismate synthase and 4-amino-4-deoxychorismate lyase were linked under the control of gap promoter, and the gene encoding wild-type HFM122 was further linked under the control of tu promoter.

(d) Preparation of Other Plasmids

A DNA fragment for a vector was synthesized by PCR using the plasmid pECsf_gapS_pabABC_tuD_HFM122 obtained above as a template and using primers pGapABA_tu vec F (SEQ ID NO: 27, GGAGGTTTAAACAAGCGG) and pGapABA_tu vec R (SEQ ID NO: 28, AATTTAGATCCTCCTTTGGACTTCGTG). Subsequently, a plasmid containing a gene (SEQ ID NO: 3, 5, or 7) encoding each polypeptide having 4-aminobenzoic acid hydroxylation activity was prepared by artificial gene synthesis, and a DNA fragment for an insert was synthesized by PCR using this plasmid as a template and using primers shown in the column “Primer” of Table B1. These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid shown in the column “Plasmid” of Table B1. The ECOS Competent E. coli DH5α strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 26, GCTTGTTAGATATCTTGAAATCGGCTTTC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmids, the genes encoding 4-amino-4-deoxychorismate synthase and 4-amino-4-deoxychorismate lyase were linked under the control of gap promoter, and the gene encoding wild-type hydroxylase was further linked under the control of tu promoter.

TABLE B1

| | | | SEQ |
|---|---|---|---|
| | | | ID |
| Plasmid | Primer | Sequence (5′-→3′) | NO |
| pECsf_ | HFM77 | AGGAGGATCTAAATTATG | 29 |
| gapS_ | ins F | CGTACTCAGGTGGGAATC | |
| pabABC_ | HFM77 | CTTGTTTAAACCTCCTTA | 30 |
| tu_HFM77 | ins R | AGCAAGTGGCATGC | |
| pECsf_ | HFM339 | AGGAGGATCTAAATTATG | 31 |
| gapS_ | ins F | CGCACTCAGGTGGCAATC | |
| pabABC_ | HFM339 | CTTGTTTAAACCTCCTTA | 32 |
| tu_HFM339 | ins R | GTATGGCAGGCCTACG | |
| pECsf_ | HFM388 | AGGAGGATCTAAAHATGC | 33 |
| gapS_ | ins F | GCACCCAAGTGGTCATC | |
| pabABC_ | HFM388 | CTTGTTTAAACCTCCTTAG | 34 |
| tu_HFM388 | ins R | AACGGCAGACCCACGTAG | |

(2) Preparation of Plasmid Containing Gene Encoding Mutant Enzyme

The preparation of a plasmid containing a gene encoding a mutant enzyme will be given below by taking, as an example, the preparation of a plasmid containing a gene encoding a mutant enzyme in which tyrosine at position 201 of HFM77 was substituted with phenylalanine.

A plasmid pECsf_gapS_pabABC_tu_HFM77 Y201F was constructed by PCR using a plasmid pECsf_gapS_pabABC_tu_HFM77 as a template and using complementary primers HFM77 Y201F F (SEQ ID NO: 51, CTCATCTTCGCACATCACGACCGCGGA) and HFM77 Y201F R (SEQ ID NO: 52, ATGTGCGAAGATGAGCTCTTCGGATGA). The PCR product was treated with DpnI (Takara Bio Inc.). The ECOS Competent E. coli DH5α strain (Nippon Gene Co., Ltd.) was transformed with the solution thus treated. The cell solution was spread over LBKm agar medium and then left standing overnight at 37° C. The obtained colonies were selected as a transformant. The transformant was inoculated to 2 mL of LBKm liquid medium and cultured overnight at 37° C. A plasmid was purified from this culture solution using NucleoSpin Plasmid EasyPure (Takara Bio Inc.).

In a similar manner, a plasmid containing a gene encoding each enzyme mutant was obtained by PCR using a plasmid shown in the column “Template” of Table B2 instead of the plasmid pECsf_gapS_pabABC_tu_HFM77 and using primers shown in the column “Primer” of Table B2 instead of the primers HFM77 Y201F F and HFM77 Y201F R.

TABLE B2

| | | | SEQ |
|---|---|---|---|
| | | | ID |
| Template | Primer | Sequence (5′-→3′) | NO |
| pECsf_gapS_ | HFM77 | CGCATGTTCTTCCAGTGCGC | 53 |
| pabABC_ | Y222F F | ACCTACC | |
| tu_HFM77 | HFM77 | CTGGAAGAACATGCGCTGGA | 54 |
| | Y222F R | TATTCGG | |
| pECsf_gapS_ | HFM122 | TTGATCTTCTCGAACCATGA | 55 |
| pabABC_ | Y201F F | TCGCGGT | |
| tuD_HFM122 | HFM122 | GTTCGAGAAGATCAACTCGT | 56 |
| | Y201F R | GGTCACA | |
| pECsf_gapS_ | HFM122 | CGCTATTTCGTGCAGTGCTC | 57 |
| pabABC_ | Y222F F | ACTCGAC | |
| tuD_HFM122 | HFM122 | CTGCACGAAATAGCGGGAGC | 58 |
| | Y222F R | GTGTCGG | |
| pECsf_gapS_ | HFM339 | CTGATCTTCGTCAACCACGA | 59 |
| pabABC_ | Y201F F | CCGAGGC | |
| tu_HFM339 | HFM339 | GTTGACGAAGATCAGTTCTG | 60 |
| | Y201F R | GGCTGAC | |
| pECsf_gapS_ | HFM339 | CGGTACTTCGTCCAATGCCC | 61 |
| pabABC_ | Y222F F | TTTGACC | |
| tu_HFM339 | HFM339 | TTGGACGAAGTACCGTGAAC | 62 |
| | Y222F R | GGTGCAT | |
| pECsf_gapS_ | HFM388 | CTCGTGTTCGCTAATCACCC | 63 |
| pabABC_ | Y201F F | ACGCGGG | |
| tu_HFM388 | HFM388 | ATTAGCGAACACGAGTTCAT | 64 |
| | Y201F R | GATCGAC | |
| pECsf_gapS_ | HFM388 | CGCTACTTCATCCAGTGCCC | 65 |
| pabABC_ | Y222F F | TTTGGAG | |
| tu_HFM388 | HFM388 | CTGGATGAAGTAGCGAGAAC | 66 |
| | Y222F R | GGGTATG | |

(3) Introduction of Plasmid into Host Cell

The Corynebacterium glutamicum DRHG145 strain (see Japanese Patent Application No. 2014-523757) was transformed with each plasmid obtained above by electroporation (Bio-Rad Laboratories, Inc.). The obtained transformed cell solution was spread over LBKm agar medium and then left standing at 30° C. for 2 days. The obtained colonies were used as a transformant.

(4) Culture of Transformant

Each transformant obtained above was inoculated to 1 mL of CGYE medium (containing 50 μg/mL kanamycin sulfate) shown in Table B3, and cultured overnight at 30° C. 100 μL of the obtained culture solution was inoculated to 10 mL of CGXII medium (containing 50 μg/mL kanamycin sulfate) shown in Table B4, and cultured at 30° C. for approximately 48 hours. Then, bacterial cells were removed by centrifugation to obtain a culture supernatant. The concentration of 4-amino-3-hydroxybenzoic acid in the obtained culture supernatant was quantified in accordance with the method of Reference Example 1. The rate of improvement in the ability to produce 4-amino-3-hydroxybenzoic acid was calculated according to the equation given below. In the equation, “WT” represents a “transformant harboring the plasmid containing the gene encoding the wild-type enzyme”, and “MT” represents a “transformant harboring the plasmid containing the gene encoding the mutant enzyme and prepared from the plasmid containing the gene encoding the wild-type enzyme”.

Rate of improvement in production ability=Ability of MT to produce 4-amino-3-hydroxybenzoic acid/Ability of WT to produce 4-amino-3-hydroxybenzoic acid (Equation 1)

TABLE B3
CGYE medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Yeast extract | 1 | g |

TABLE B4
CGXII medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Tryptone | 10 | g |

(5) Results

As shown in Table B5, the bacterial strain harboring each mutant enzyme had the more improved ability to produce 4-amino-3-hydroxybenzoic acid than the bacterial strain harboring the wild-type enzyme.

TABLE B5

| | Ability to produce 4- | |
|---|---|---|
| | amino-3-hydroxybenzoic | Rate of improvement |
| Hydroxylase | acid (g/L) | in production ability |
| HFM77 wt | 0.073 | 1.00 |
| HFM77 Y201F | 0.114 | 1.56 |
| HFM77 Y222F | 0.100 | 1.37 |
| HFM122 wt | 0.134 | 1.00 |
| HFM122 Y201F | 0.224 | 1.67 |
| HFM122 Y222F | 0.256 | 1.90 |
| HFM339 wt | 0.016 | 1.00 |
| HFM339 Y201F | 0.061 | 3.80 |
| HFM339 Y222F | 0.139 | 8.74 |
| HFM388 wt | 0.033 | 1.00 |
| HFM388 Y201F | 0.079 | 2.38 |
| HFM388 Y222F | 0.230 | 6.95 |

### Example C1 Production of 4-Amino-3-Hydroxybenzoic Acid

In the following Example, PCR was performed using PrimeSTAR Max Premix (Takara Bio Inc.).

(1) Preparation of Plasmid Containing Gene Encoding Wild-Type Enzyme

(a) Preparation of Plasmid pECsf_gapS_pabABC

A DNA fragment containing genes encoding 4-amino-4-deoxychorismate synthase and 4-amino-4-deoxychorismate lyase was amplified by PCR using the genome extracted from the Corynebacterium glutamicum ATCC13032 strain by a routine method as a template and using primers GN14_127 (SEQ ID NO: 13, TATTAATTAAATGCGCGTTTTAATTATTGATAATTATGATTC) and GN14_133 (SEQ ID NO: 14, TTGCGGCCGCTTGTTTAAACCTCCTTACAGAAAAATGGTTGGGCG). This fragment was inserted between the PacI site and the NotI site of a plasmid pECsf_gapS (see Japanese Patent Application No. 2015-25491) to obtain a plasmid pECsf_gapS_pabABC.

(b) Preparation of Plasmid pECsf_gapS_pabABC_HFM122

A DNA fragment for a vector was synthesized by PCR using the plasmid pECsf_gapS_pabABC obtained above as a template and using primers pabABCcory vec R (SEQ ID NO: 15, AAATTTAAACCTCCTTTACAGAAAAATGGTTGG) and pabABCcory vec F (SEQ ID NO: 16, GGAGGTTTAAACAAGCGGCCGCGATATC). Subsequently, a plasmid containing a gene (SEQ ID NO: 1) encoding a polypeptide HFM122 having 4-aminobenzoic acid hydroxylation activity was prepared by artificial gene synthesis, and a DNA fragment for an insert was synthesized by PCR using this plasmid as a template and using primers pECsfD HFM122 F (SEQ ID NO: 17, AGGAGGTTTAAATTTATGCGCACTCAGGTGGCTAT) and pECsfD HFM122 R (SEQ ID NO: 18, CTTGTTTAAACCTCCTTATACGAGTGGCAGTCCTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_HFM122. The ECOS Competent E. coli DH5α strain (Nippon Gene Co., Ltd.) was transformed with the obtained plasmid solution. The cell solution was spread over LBKm agar medium (1% Bacto Tryptone, 0.5% yeast extract, 1% NaCl, 50 μg/mL kanamycin sulfate, 1.5% agar) and then left standing overnight at 37° C. The obtained colonies were subjected to PCR reaction using Sapphire Amp (Takara Bio Inc.) and primers pabABC+pobA for CPCR F (SEQ ID NO: 19, GCTATCAAAACATTCGGCACATTGGTTTTCC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium (1% Bacto Tryptone, 0.5% yeast extract, 1% NaCl, 50 μg/mL kanamycin sulfate) and cultured overnight at 37° C. A plasmid was purified from this culture solution using NucleoSpin Plasmid EasyPure (Takara Bio Inc.).

(c) Preparation of Plasmid

pECsf_gapS_pabABC_tuD_HFM122

A DNA fragment for a vector was synthesized by PCR using the plasmid pECsf_gapS_pabABC_HFM122 obtained above as a template and using primers pabC last R (SEQ ID NO: 21, TTACAGAAAAATGGTTGGGCGCAA) and HFM122 F (SEQ ID NO: 22, ATGCGCACTCAGGTGGCTATCG). Subsequently, a DNA fragment (SEQ ID NO: 23, TACGTACCTGCAGGTAGCGTGTCAGTAGGCGCGTAGGGTAAGTGGGGTAGCGGCTTG TTAGATATCTTGAAATCGGCTTTCAACAGCATTGATTTCGATGTATTTAGCTGGCCG TTACCCTGCGAATGTCCACAGGGTAGCTGGTAGTTTGAAAATCAACGCCGTTGCCCT TAGGATTCAGTAACTGGCACATTTTGTAATGCGCTAGATCTGTGTGCTCAGTCTTCC AGGCTGCTTATCACAGTGAAAGCAAAACCAATTCGTGGCTGCGAAAGTCGTAGCCAC CACGAAGTCCAAAGGAGGATCTAAATTATGAATAATATAAAAGGAGGAATTAATTAA) containing tuf gene (cg0587) promoter (hereinafter, referred to as tu promoter) carried by the Corynebacterium glutamicum ATCC13032 strain was prepared by artificial gene synthesis, and a DNA fragment for an insert was synthesized by PCR using this fragment as a template and using primers pabC-Ptu F (SEQ ID NO: 24, ACCATTTTTCTGTAATACGTACCTGCAGGTAGCGTG) and Ptu-HFM122 R (SEQ ID NO: 25, CACCTGAGTGCGCATTTAATTAATTCCTCCTTTTA). These PCR products were treated with DpnI (Takara Bio Inc.). Then, the respective DNA fragments were purified using NucleoSpin Gel and PCR Clean-up (Takara Bio Inc.) and ligated using In-Fusion HD Cloning Kit (Takara Bio Inc.) to construct a plasmid pECsf_gapS_pabABC_tuD_HFM122. The ECOS Competent E. coli DH5α strain (Nippon Gene Co., Ltd.) was transformed with the obtained plasmid solution. The cell solution was spread over LBKm agar medium and then left standing overnight at 37° C. The obtained colonies were subjected to PCR reaction using Sapphire Amp (Takara Bio Inc.) and primers Ptu seq 1 (SEQ ID NO: 26, GCTTGTTAGATATCTTGAAATCGGCTTTC) and pabABC+pobA for CPCR R (SEQ ID NO: 20, GGAAGATGCGTGATCTGATCCTTCAACTC) to select a transformant confirmed to harbor the DNA fragment of interest. The obtained transformant was inoculated to 2 mL of LBKm liquid medium and cultured overnight at 37° C. A plasmid was purified from this culture solution using NucleoSpin Plasmid EasyPure (Takara Bio Inc.).

In the constructed plasmid, the genes encoding 4-amino-4-deoxychorismate synthase and 4-amino-4-deoxychorismate lyase were linked under the control of gap promoter, and the gene encoding wild-type HFM122 was further linked under the control of tu promoter.

(2) Preparation of Plasmid Containing Gene Encoding Mutant Enzyme

The preparation of a plasmid containing a gene encoding a mutant enzyme will be given below by taking, as an example, the preparation of a plasmid containing a gene encoding a mutant enzyme in which valine at position 47 of HFM122 was substituted with isoleucine.

A plasmid pECsf_gapS_pabABC_tuD_HFM122_V47I was constructed by PCR using a plasmid pECsf_gapS_pabABC_tuD_HFM122 as a template and using complementary primers HFM122 V47I F (SEQ ID NO: 67, GCTGGTATTCTGGAACGTATCACGGTG) and HFM122 V47I R (SEQ ID NO: 68, TTCCAGAATACCAGCCCGAACTCGGCC). The PCR product was treated with DpnI (Takara Bio Inc.). The ECOS Competent E. coli DH5α strain (Nippon Gene Co., Ltd.) was transformed with the solution thus treated. The cell solution was spread over LBKm agar medium and then left standing overnight at 37° C. The obtained colonies were selected as a transformant. The transformant was inoculated to 2 mL of LBKm liquid medium and cultured overnight at 37° C. A plasmid was purified from this culture solution using NucleoSpin Plasmid EasyPure (Takara Bio Inc.).

In a similar manner, a plasmid containing a gene encoding each enzyme mutant was obtained by PCR using primers shown in the column “Primer” of Table C1 instead of the primers HFM122 V47I F and HFM122 V47I R.

TABLE C1

| | | | SEQ |
|---|---|---|---|
| Plasmid of | | | ID |
| interest | Primer | Sequence (5′-→3′) | NO |
| pECsf_gapS_ | HFM122 | GCTGGTTCTCTGGAACGTATCACGGTG | 69 |
| pabABC_tuD_ | V47S F | | |
| HFM122_V47S | HFM122 | TTCCAGAGAACCAGCCCGAACTCGGCC | 70 |
| | V47S R | | |
| pECsf_gapS_ | HFM122 | GCTGGTACACTGGAACGTATCACGGTG | 71 |
| pabABC_tuD_ | V47T F | | |
| HFM122_V47T | HFM122 | TTCCAGTGTACCAGCCCGAACTCGGCC | 72 |
| | V47T R | | |
| pECsf_gapS_ | HFM122 | GCTGGTTGTCTGGAACGTATCACGGTG | 73 |
| pabABC_tuD_ | V47C F | | |
| HFM122_V47C | HFM122 | TTCCAGAGAACCAGCCCGAACTCGGCC | 74 |
| | V47C R | | |
| pECsf_gapS_ | HFM122 | GCTGGTATGCTGGAACGTATCACGGTG | 75 |
| pabABC_tuD_ | V47M F | | |
| HFM122_V47M | HFM122 | TTCCAGCATACCAGCCCGAACTCGGCC | 76 |
| | V47M R | | |
| pECsf_gapS_ | HFM122 | GCTGGTCAACTGGAACGTATCACGGTG | 77 |
| pabABC_tuD_ | V47Q F | | |
| HFM122_V47Q | HFM122 | TTCCAGTTGACCAGCCCGAACTCGGCC | 78 |
| | V47Q R | | |
| pECsf_gapS_ | HFM122 | TTGGTGGCAGCTGGCGCTAATCTTGCG | 79 |
| pabABC_tuD_ | H72A F | | |
| HFM122_H72A | HAM122 | GCCAGCTGCCACCAAGCCCTCTCGGCG | 80 |
| | H72A R | | |
| pECsf_gapS_ | HFM122 | TTGGTGATGGCTGGCGCTAATCTTGCG | 81 |
| pabABC_tuD_ | H72M F | | |
| HFM122_H72M | HFM122 | GCCAGCCATCACCAAGCCCTCTCGGCG | 82 |
| | H72M R | | |
| pECsf_gapS_ | HFM122 | TTTGCCATGGCGTCGATGCGCTCACCG | 83 |
| pabABC_tuD_ | L210M F | | |
| HFM122_ | HFM122 | CGACGCCATGGCAAAACCGCGATCATG | 84 |
| L210M | L210M R | | |
| pECsf_gapS_ | HFM122 | CCACCCGCAGGAGCGAAAGGGATGAAC | 85 |
| pabABC_tuD_ | T294A F | | |
| HFM122_ | HFM122 | CGCTCCTGCGGGTGGAACGATATGAGC | 86 |
| T294A | T294A R | | |
| pECsf_gapS_ | HFM122 | CCACCCGGTGGAGCGAAAGGGATGAAC | 87 |
| pabABC_tuD_ | T294G F | | |
| HFM122_ | HFM122 | CGCTCCACCGGGTGGAACGATATGAGC | 88 |
| 7294G | T294G R | | |
| pECsf_gapS_ | HFM122 | CCACCCTGTGGAGCGAAAGGGATGAAC | 89 |
| pabABC_tuD_ | T294C F | | |
| HFM122_ | HFM122 | CGCTCCACAGGGTGGAACGATATGAGC | 90 |
| T294C | T294C R | | |
| pECsf_gapS_ | HFM122 | CCACCCTCTGGAGCGAAAGGGATGAAC | 91 |
| pabABC_tuD_ | T294S F | | |
| HFM122_ | HFM122 | CGCTCCAGAGGGTGGAACGATATGAGC | 92 |
| T294S | T294S R | | |
| pECsf_gapS_ | HFM122 | GAGAACGTTGTAGGACTGCCACTCGTA | 93 |
| pabAB_tuD_ | Y385V F | | |
| HFM122_ | HFM122 | TCCTACAACGTTCTCCGCCAGGGTGAC | 94 |
| Y385V | Y385V R | | |
| pECsf_gapS_ | HFM122 | GAGAACCTCGTAGGACTGCCACTCGTA | 95 |
| pabABC_tUD_ | Y385L F | | |
| HFM122_ | HFM122 | TCCTACGAGGTTCTCCGCCAGGGTGAC | 96 |
| Y385L | Y385L R | | |
| pECsf_gapS_ | HFM122 | GAGAACATTGTAGGACTGCCACTCGTA | 97 |
| pabABC_tuD_ | Y385I F | | |
| HFM122_ | HFM122 | TCCTACAATGTTCTCCGCCAGGGTGAC | 98 |
| Y385I | Y385I R | | |
| pECsf_gapS_ | HFM122 | GAGAACATGGTAGGACTGCCACTCGTA | 99 |
| pabABC_tuD_ | Y385M F | | |
| HFM122_ | HFM122 | TCCTACCATGTTCTCCGCCAGGGTGAC | 100 |
| Y385M | Y385M R | | |

(3) Introduction of Plasmid into Host Cell

The Corynebacterium glutamicum DRHG145 strain (see Japanese Patent Application No. 2014-523757) was transformed with each plasmid obtained above by electroporation (Bio-Rad Laboratories, Inc.). The obtained transformed cell solution was spread over LBKm agar medium and then left standing at 30° C. for 2 days. The obtained colonies were used as a transformant.

(4) Culture of Transformant

Each transformant obtained above was inoculated to 1 mL of CGYE medium (containing 50 μg/mL kanamycin sulfate) shown in Table C2, and cultured overnight at 30° C. 100 μL of the obtained culture solution was inoculated to 10 mL of CGXII medium (containing 50 μg/mL kanamycin sulfate) shown in Table C3, and cultured at 30° C. for approximately 48 hours. Then, bacterial cells were removed by centrifugation to obtain a culture supernatant. The concentration of 4-amino-3-hydroxybenzoic acid in the obtained culture supernatant was quantified in accordance with the method of Reference Example 1. The rate of improvement in the ability to produce 4-amino-3-hydroxybenzoic acid was calculated according to the equation given below. In this context, “WT” represents a “transformant harboring the plasmid containing the gene encoding the wild-type enzyme”, and “MT” represents a “transformant harboring the plasmid containing the gene encoding the mutant enzyme and prepared from the plasmid containing the gene encoding the wild-type enzyme”.

Rate of improvement in production ability=Ability of MT to produce 4-amino-3-hydroxybenzoic acid/Ability of WT to produce 4-amino-3-hydroxybenzoic acid (Equation 1)

TABLE C2
CGYE medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Yeast extract | 1 | g |

TABLE C3
CGXII medium composition (per L)

| | | |
|---|---|---|
| Glucose | 50 | g |
| (NH₄)₂SO₄ | 20 | g |
| Urea | 5 | g |
| KH₂PO₄ | 1 | g |
| K₂HPO₄ | 1 | g |
| MgSO₄•7H₂O | 0.25 | g |
| CaCl₂•2H₂O | 10 | mg |
| FeSO₄•7H₂O | 10 | mg |
| MnSO₄•5H₂O | 10 | mg |
| ZnSO₄•7H₂O | 1 | mg |
| CuSO₄•5H₂O | 0.2 | mg |
| NiCl₂•6H₂O | 0.02 | mg |
| Biotin (pH 7) | 0.2 | mg |
| Tryptone | 10 | g |

(5) Results

As shown in Table C4, the bacterial strain harboring each mutant enzyme had the more improved ability to produce 4-amino-3-hydroxybenzoic acid than the bacterial strain harboring the wild-type enzyme.

TABLE C4

| | Ability to produce 4- | |
|---|---|---|
| | amino-3-hydroxybenzoic | Rate of improvement |
| Hydroxylase | acid (g/L) | in production ability |
| HFM122 wt | 0.134 | 1.00 |
| HFM122 V47I | 0.306 | 2.28 |
| HFM122 V47S | 0.163 | 1.21 |
| HFM122 V47T | 0.202 | 1.50 |
| HFM122 V47C | 0.174 | 1.30 |
| HFM122 V47M | 0.290 | 2.16 |
| HFM122 V47Q | 0.254 | 1.89 |
| HFM122 H72A | 0.164 | 1.22 |
| HFM122 H72M | 0.216 | 1.61 |
| HFM122 L210M | 0.173 | 1.29 |
| HFM122 T294A | 0.160 | 1.19 |
| HFM122 T294G | 0.187 | 1.39 |
| HFM122 T294C | 0.190 | 1.41 |
| HFM122 T294S | 0.378 | 2.81 |
| HFM122 Y385V | 0.157 | 1.17 |
| HFM122 Y385L | 0.168 | 1.25 |
| HFM122 Y385I | 0.186 | 1.38 |
| HFM122 Y385M | 0.167 | 1.24 |

### Reference Example 1 Quantification of 4-Amino-3-Hydroxybenzoic Acid

4-Amino-3-hydroxybenzoic acid was quantified by HPLC. A reaction solution to be subjected to HPLC analysis was appropriately diluted with 0.1 phosphoric acid. Then, insoluble matter was removed using AcroPrep 96-well filter plates (0.2 μm GHP membrane, Nihon Pall Ltd.).

The HPLC apparatus used was Chromaster (Hitachi High-Tech Science Corp.). The analytical column used was L-column ODS (4.6 mm I.D.×150 mm, Chemicals Evaluation and Research Institute, Japan). Eluent A was a 0.1% phosphoric acid solution of 0.1 M potassium dihydrogen phosphate, and eluent B was 70% methanol. Gradient elution was performed under conditions involving a flow rate of 1.0 mL/min and a column temperature of 40° C. A UV detector (detection wavelength: 280 nm) was used for the detection of 4-amino-3-hydroxybenzoic acid. A concentration calibration curve was prepared using a standard sample [4-amino-3-hydroxybenzoic acid (distributor code A1194, Tokyo Chemical Industry Co., Ltd.)]. 4-Amino-3-hydroxybenzoic acid was quantified on the basis of the concentration calibration curve.

What is claimed is:

1. A polypeptide having 4-aminobenzoic acid hydroxylation activity, the polypeptide being selected from the group consisting of the following A) to C):
A) a polypeptide having 4-aminobenzoic acid hydroxylation activity, consisting of the amino acid sequence represented by SEQ ID NO: 2 or an amino acid sequence having at least 47% identity thereto, and having an amino acid residue at position 47 of the amino acid sequence represented by SEQ ID NO: 2 or a position corresponding thereto being leucine;
B) a polypeptide having 4-aminobenzoic acid hydroxylation activity, consisting of the amino acid sequence represented by SEQ ID NO: 2 or an amino acid sequence having at least 51% identity thereto, and having an amino acid residue at position 201 or 222 of the amino acid sequence represented by SEQ ID NO: 2, or a position corresponding to the position 201 or 222 being phenylalanine; and
C) a polypeptide having 4-aminobenzoic acid hydroxylation activity, consisting of the amino acid sequence represented by SEQ ID NO: 2 or an amino acid sequence having at least 90% identity thereto, and having an amino acid residue at position 47, 72, 210, 294 or 385 of the amino acid sequence represented by SEQ ID NO: 2, or a position corresponding to the position 47, 72, 210, 294 or 385 being the following amino acid:
(a) the position 47 or the position corresponding thereto: isoleucine, serine, threonine, cysteine, methionine, or glutamine,
(b) the position 72 or the position corresponding thereto: alanine or methionine,
(c) the position 210 or the position corresponding thereto: methionine,
(d) the position 294 or the position corresponding thereto: alanine, glycine, cysteine, or serine, and
(e) the position 385 or the position corresponding thereto: valine, leucine, isoleucine, or methionine.

2. A method for producing a mutant polypeptide having 4-aminobenzoic acid hydroxylation activity, comprising substituting an amino acid residue, the substituting is selected from the group consisting of the following A′) to C′):
A′) in a polypeptide consisting of the amino acid sequence represented by SEQ ID NO: 2 or an amino acid sequence having at least 47% identity thereto, and having 4-aminobenzoic acid hydroxylation activity, substitution of an amino acid residue at position 47 of the amino acid sequence represented by SEQ ID NO: 2 or a position corresponding thereto with leucine;
B′) in a polypeptide consisting of the amino acid sequence represented by SEQ ID NO: 2 or an amino acid sequence having at least 51% identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and
C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:
(a) the position 47 or the position corresponding thereto: isoleucine, serine, threonine, cysteine, methionine, or glutamine,
(b) the position 72 or the position corresponding thereto: alanine or methionine,
(c) the position 210 or the position corresponding thereto: methionine,
(d) the position 294 or the position corresponding thereto: alanine, glycine, cysteine, or serine, and
(e) the position 385 or the position corresponding thereto: valine, leucine, isoleucine, or methionine.

3. A method for improving 4-aminobenzoic acid hydroxylation activity, comprising substituting an amino acid residue, the substituting is selected from the group consisting of the following A′) to C′):
A′) in a polypeptide consisting of the amino acid sequence represented by SEQ ID NO: 2 or an amino acid sequence having at least 47% identity thereto, and having 4-aminobenzoic acid hydroxylation activity, substitution of an amino acid residue at position 47 of the amino acid sequence represented by SEQ ID NO: 2 or a position corresponding thereto with leucine;
B′) in a polypeptide consisting of the amino acid sequence represented by SEQ ID NO: 2 or an amino acid sequence having at least 51% identity thereto, and having 4-aminobenzoic acid hydroxylation activity, substitution of an amino acid residue at position 201 or 222 of the amino acid sequence represented by SEQ ID NO: 2, or a position corresponding to the position 201 or 222 with phenylalanine; and
C′) in a polypeptide consisting of the amino acid sequence represented by SEQ ID NO: 2 or an amino acid sequence having at least 90% identity thereto, and having 4-aminobenzoic acid hydroxylation activity, substitution of an amino acid residue at position 47, 72, 210, 294 or 385 of the amino acid sequence represented by SEQ ID NO: 2, or a position corresponding to the position 47, 72, 210, 294 or 385 with the following amino acid:
(a) the position 47 or the position corresponding thereto: isoleucine, serine, threonine, cysteine, methionine, or glutamine,
(b) the position 72 or the position corresponding thereto: alanine or methionine,
(c) the position 210 or the position corresponding thereto: methionine,
(d) the position 294 or the position corresponding thereto: alanine, glycine, cysteine, or serine, and
(e) the position 385 or the position corresponding thereto: valine, leucine, isoleucine, or methionine.

4. The method according to claim 2, wherein the substitution of an amino acid residue in A′) is substitution of valine with leucine, and the substitution of an amino acid residue in B′) is substitution of tyrosine with phenylalanine.

5. A polynucleotide encoding the polypeptide according to claim 1.

6. A vector or a DNA fragment comprising the polynucleotide according to claim 5.

7. A transformed cell comprising the vector or the DNA fragment according to claim 6.

8. The transformed cell according to claim 7, wherein the transformed cell is derived from E. coli or a bacterium of the genus Corynebacterium.

9. The transformed cell according to claim 7, wherein
the transformed cell is a microorganism capable of supplying 4-aminobenzoic acids.

10. A method for producing 4-amino-3-hydroxybenzoic acids, comprising a step of culturing a transformed cell according to claim 7.

11. The method according to claim 10, further comprising a step of collecting 4-amino-3-hydroxybenzoic acids from a culture medium.

12. The method according to claim 10, wherein the culturing is performed in the presence of 4-aminobenzoic acids.

13. The method according to claim 10, wherein the 4-amino-3-hydroxybenzoic acids are 4-amino-3-hydroxybenzoic acid derivatives of the following formula (1): 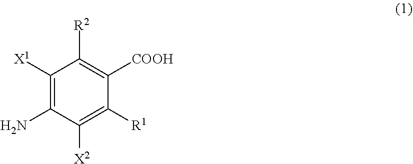
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, R² represents a hydrogen atom, a hydroxy group, a methoxy group, an amino group, a fluorine atom, a chlorine atom, a bromine atom, an iodine atom, a carboxy group, a methyl group, or an ethyl group, and X¹ and X² each represent a hydrogen atom or a hydroxy group, at least one of which represents a hydroxy group, and
the 4-aminobenzoic acids are 4-aminobenzoic acid derivatives of the following formula (2): 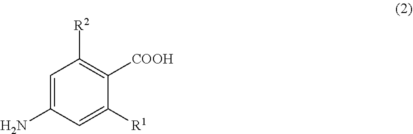
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, and R² represents a hydrogen atom, a hydroxy group, a methoxy group, an amino group, a fluorine atom, a chlorine atom, a bromine atom, an iodine atom, a carboxy group, a methyl group, or an ethyl group.

14. The method according to claim 3, wherein the substitution of the amino acid residue in A′ is substitution of valine with leucine, and the substitution of the amino acid residue in B′ is substitution of tyrosine with phenylalanine.

15. The transformed cell according to claim 8, wherein the transformed cell is a microorganism capable of supplying 4-aminobenzoic acids.

16. A method for producing 4-amino-3-hydroxybenzoic acids, comprising a step of culturing a transformed cell according to claim 8.

17. A method for producing 4-amino-3-hydroxybenzoic acids, comprising a step of culturing a transformed cell according to claim 15.

18. The method according to claim 17, further comprising a step of collecting 4-amino-3-hydroxybenzoic acids from a culture medium.

19. The method according to claim 17, wherein the 4-amino-3-hydroxybenzoic acids are 4-amino-3-hydroxybenzoic acid derivatives of the following formula (1): 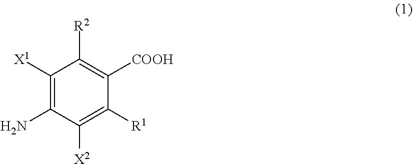
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, R² represents a hydrogen atom, a hydroxy group, a methoxy group, an amino group, a fluorine atom, a chlorine atom, a bromine atom, an iodine atom, a carboxy group, a methyl group, or an ethyl group, and X¹ and X² each represent a hydrogen atom or a hydroxy group, at least one of which represents a hydroxy group, and
the 4-aminobenzoic acids are 4-aminobenzoic acid derivatives of the following formula (2): 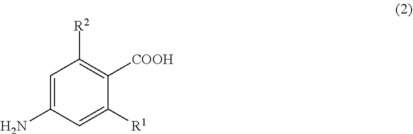
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, and R² represents a hydrogen atom, a hydroxy group, a methoxy group, an amino group, a fluorine atom, a chlorine atom, a bromine atom, an iodine atom, a carboxy group, a methyl group, or an ethyl group.

20. The method according to claim 18, wherein the 4-amino-3-hydroxybenzoic acids are 4-amino-3-hydroxybenzoic acid derivatives of the following formula (1): 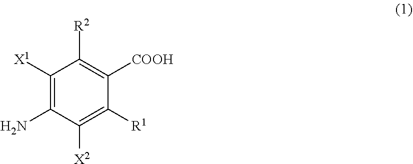
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, R² represents a hydrogen atom, a hydroxy group, a methoxy group, an amino group, a fluorine atom, a chlorine atom, a bromine atom, an iodine atom, a carboxy group, a methyl group, or an ethyl group, and X¹ and X² each represent a hydrogen atom or a hydroxy group, at least one of which represents a hydroxy group, and
the 4-aminobenzoic acids are 4-aminobenzoic acid derivatives of the following formula (2): 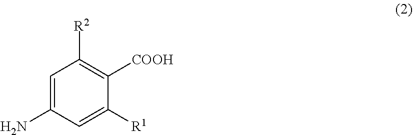
wherein R¹ represents a hydrogen atom, a hydroxy group, a methoxy group, an amino group, a fluorine atom, a chlorine atom, a bromine atom, an iodine atom, a carboxy group, a methyl group, or an ethyl group, and R² represents a hydrogen atom, a hydroxy group, a methoxy group, an amino group, a fluorine atom, a chlorine atom, a bromine atom, an iodine atom, a carboxy group, a methyl group, or an ethyl group.
